(19) United States
(12) Patent Application Publication
(10) Pub. No.: US 2024/0142461 A1
(43) Pub. Date: May 2, 2024

(54) WATER LOSS FROM SILICONE TUBING AND EFFECT ON PROTEIN CONCENTRATION DURING DRUG PRODUCT MANUFACTURING

(71) Applicant: Regeneron Pharmaceuticals, Inc., Tarrytown, NY (US)

(72) Inventors: Sonia Bedi, Basking Ridge, NJ (US); Pinaki Ranadive, Elmsford, NY (US)

(21) Appl. No.: 18/379,467

(22) Filed: Oct. 12, 2023

Related U.S. Application Data
(60) Provisional application No. 63/421,103, filed on Oct. 31, 2022.

(51) Int. Cl. G01N 33/68; C12M 1/12; C12M 1/34

(52) U.S. Cl. CPC G01N 33/6803; C12M 23/06; C12M 41/32; C12M 41/44

(57) ABSTRACT

The present invention generally pertains to methods of predicting water loss from a sample. In particular, the present invention pertains to modeling water loss from a drug substance in silicone tubing based on the properties of the tubing and the drug substance.

### CROSS-REFERENCE TO RELATED APPLICATIONS

This application claims the benefit of U.S. Provisional Application No. 63/421,103, filed Oct. 31, 2022, which is incorporated by reference herein in its entirety.

### FIELD

This application relates to methods for predicting water loss from a sample in silicone tubing.

### BACKGROUND

Drug substance (DS) encounters a variety of materials during drug product (DP) manufacturing or fill/finish operation, and it is important to evaluate physicochemical compatibility of DS with these contact parts. Silicone tubing is one such elastomeric component used during DP manufacturing to facilitate fluid transfer and filling operation. Silicone tubing has known permeability to various low molecular weight species and gases, and it has been qualitatively shown that water loss occurs from silicone tubing. However, the potential impact of water loss from silicone tubing and other polymeric tubings on drug and/or excipient concentration in the context of DP manufacturing has not been quantified or thoroughly investigated to date. Controlling drug and excipient concentration is a vital consideration in the manufacturing process, and therefore understanding how the interaction of tubing characteristics and drug substance parameters affects water loss and concentration change is important for designing more representative process characterization and development studies for DP tech transfer.

Therefore, demand exists for methods to predict and prevent water loss and concentration change in drug substance stored in tubing.

### SUMMARY

A method has been developed for predicting water loss from a sample in tubing, for example a drug substance in silicone tubing. This disclosure describes that a linear relationship exists between water loss and concentration change of a protein in a drug substance; therefore, a method has further been developed for predicting the concentration change of a protein in a drug substance in tubing. When taking into consideration a range of acceptable concentration change of a protein, a method has additionally been developed for selecting a range of acceptable hold times for a sample in tubing. Because rate of water loss is dependent on the characteristics of a tubing, for example the internal diameter (ID), thickness of the tubing wall, and/or surface area-to-volume ratio, a method has also been developed for selecting a tubing using a model of predicted water loss of a sample.

This disclosure provides a method for predicting water loss from a sample including a protein at a time point. In some exemplary embodiments, the method comprises (a) obtaining a sample including a protein stored in tubing; (b) using said sample and said tubing to generate a model of water lost from said sample over time in said tubing; and (c) using said model to predict an amount of water lost from said sample at a time point.

In one aspect, the model is generated using equations 17, 18 and 20.

In one aspect, the sample is cell culture fluid (CCF), harvested cell culture fluid (HCCF), any step in the downstream processing of a drug substance (DS), drug substance, or a drug product (DP).

In one aspect, the tubing internal diameter is known. In a specific aspect, the tubing internal diameter (ID) is between about 0.1 mm and about 32 mm, between about 0.2 mm and about 26 mm, between about 0.5 mm and about 16 mm, between about 0.8 mm and about 13 mm, between about 0.8 mm and about 10 mm, about 0.1 mm, about 0.2 mm, about 0.3 mm, about 0.5 mm, about 0.6 mm, about 0.8 mm, about 1 mm, about 1.2 mm, about 1.3 mm, about 1.6 mm, about 2.4 mm, about 3.2 mm, about 4.8 mm, about 6.4 mm, about 8 mm, about 9.6 mm, about 12.7 mm, about 15.9 mm, about 19 mm, about 25.4 mm, or about 31.8 mm.

In one aspect, the tubing thickness is known. In a specific aspect, the tubing thickness is between about 0.1 mm and about 10 mm, between about 0.2 mm and about 8 mm, between about 0.5 mm and about 7 mm, about 0.25 mm, about 0.4 mm, about 0.5 mm, about 0.7 mm, about 0.8 mm, about 1.6 mm, about 1.8 mm, about 2.1 mm, about 2.4 mm, about 3.2 mm, about 4 mm, about 4.8 mm, about 6.4 mm, about 7 mm, about 8 mm, about 9 mm, or about 10 mm. The size of the tubing can have any combination of ID and thickness described above and corresponding surface area-to-volume ratio.

In one aspect, the tubing surface area-to-volume ratio is known. In a specific aspect, the tubing surface area-to-volume ratio is between about 0.5 mm⁻¹ and about 5 mm⁻¹, about 0.5 mm⁻¹, about 0.83 mm⁻¹, about 1 mm⁻¹, about 1.25 mm⁻¹, about 1.26 mm⁻¹, about 1.5 mm⁻¹, about 1.54 mm⁻¹, about 1.57 mm⁻¹, about 1.67 mm⁻¹, about 2 mm⁻¹, about 2.11 mm⁻¹, about 2.5 mm⁻¹, about 2.52 mm⁻¹, about 3 mm⁻¹, about 3.1 mm⁻¹, about 3.15 mm⁻¹, about 3.33 mm⁻¹, about 3.5 mm⁻¹, about 4 mm⁻¹, about 4.12 mm⁻¹, about 4.5 mm⁻¹, about 5 mm⁻¹, about 6.25 mm⁻¹, about 6.67 mm⁻¹, about 8.0 mm⁻¹, about 13.33 mm⁻¹, about 20.0 mm⁻¹, or about 40.0 mm⁻¹,

In one aspect, at least one of the following properties is known: sample volume, concentration of excipients or density. In another aspect, the concentration of the protein is known. In a further aspect, the water activity of the sample is known. In a specific aspect, the water activity is calculated using Equations 12-15.

In one aspect, the method further comprises measuring the relative humidity. In a specific aspect, the method further comprises calculating the average relative humidity using Equation 16.

In one aspect, the time point is between 10 seconds and 168 hours, between 5 minutes and 168 hours, between 3 hours and 168 hours, between 6 hours and 240 hours, about 10 seconds, about 1 hour, about 6 hours, about 12 hours, about 24 hours, about 36 hours, about 48 hours, about 72 hours, about 96 hours, about 120 hours, about 144 hours, or about 168 hours, or about 240 hours. In one aspect, manual measurements may be taken as frequent as every five minutes to provide additional datapoints and the process may potentially be automated to continuously take measurements and make calculations in real-time.

In one aspect, the tubing is vinyl tubing, platinum-cured silicone tubing, peroxide cured silicone tubing, high-density polyethylene (HDPE) tubing, fluoropolymer tubing, thermoplastic elastomer tubing (TPE) or similar thermoplastic elastomer tubing material.

In one aspect, the method further comprises using the calculated water loss at a first time point to iteratively calculate water loss at a subsequent time point at least once. In a specific aspect, the iterative calculation is calculated using Equations 4 and 6.

In one aspect, a temperature of the tubing is between about 5° C. and about 30° C., between about 15° C. and about 30° C., between about 15° C. and about 25° C., between about 15° C. and about 21° C., between about 16° C. and about 24° C., between about 17° C. and about 23° C., between about 18° C. and about 21° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C.

In one aspect, the water loss is due to diffusive mass transfer.

This disclosure also provides a method for predicting a change in concentration of a protein at a time point. In some exemplary embodiments, the method comprises (a) obtaining a sample including a protein stored in tubing; (b) using said sample and said tubing to generate a model of change in concentration of said protein over time in said tubing; and (c) using said model to predict a change in concentration of said protein at a time point.

In one aspect, the model is generated using equations 17, 18, 20 and 21.

In one aspect, the sample is cell culture fluid (CCF), harvested cell culture fluid (HCCF), any step in the downstream processing of a drug substance (DS), drug substance, or a drug product (DP).

In one aspect, the tubing internal diameter is known. In a specific aspect, the tubing internal diameter (ID) is between about 0.1 mm and about 32 mm, between about 0.2 mm and about 26 mm, between about 0.5 mm and about 16 mm, between about 0.8 mm and about 13 mm, between about 0.8 mm and about 10 mm, about 0.1 mm, about 0.2 mm, about 0.3 mm, about 0.5 mm, about 0.6 mm, about 0.8 mm, about 1 mm, about 1.2 mm, about 1.3 mm, about 1.6 mm, about 2.4 mm, about 3.2 mm, about 4.8 mm, about 6.4 mm, about 8 mm, about 9.6 mm, about 12.7 mm, about 15.9 mm, about 19 mm, about 25.4 mm, or about 31.8 mm.

In one aspect, the tubing thickness is known. In a specific aspect, the tubing thickness is between about 0.1 mm and about 10 mm, between about 0.2 mm and about 8 mm, between about 0.5 mm and about 7 mm, about 0.25 mm, about 0.4 mm, about 0.5 mm, about 0.7 mm, about 0.8 mm, about 1.6 mm, about 1.8 mm about 2.1 mm, about 2.4 mm, about 3.2 mm, about 4 mm, about 4.8 mm, about 6.4 mm, about 7 mm, about 8 mm, about 9 mm, or about 10 mm. The size of the tubing can have any combination of ID and thickness described above and corresponding tubing surface area-to-volume ratio.

In one aspect, the tubing surface area-to-volume ratio is known. In a specific aspect, the tubing surface area-to-volume ratio is between about 0.5 mm⁻¹ and about 5 mm⁻¹, about 0.5 mm⁻¹, about 0.83 mm⁻¹, about 1 mm⁻¹, about 1.25 mm⁻¹, about 1.26 mm⁻¹, about 1.5 mm⁻¹, about 1.54 mm⁻¹, about 1.57 mm⁻¹, about 1.67 mm⁻¹, about 2 mm⁻¹, about 2.11 mm⁻¹, about 2.5 mm⁻¹, about 2.52 mm⁻¹, about 3 mm⁻¹, about 3.1 mm⁻¹, about 3.15 mm⁻¹, about 3.33 mm⁻¹, about 3.5 mm⁻¹, about 4 mm⁻¹, about 4.12 mm⁻¹, about 4.5 mm⁻¹, about 5 mm⁻¹, about 6.25 mm⁻¹, about 6.67 mm⁻¹, about 8.0 mm⁻¹, about 13.33 mm⁻¹, about 20.0 mm⁻¹, or about 40.0 mm⁻¹.

In one aspect, the sample volume is known. In another aspect, the density is known. In another aspect the concentration of the excipients is known. In another aspect, the water activity of the sample is known. In a specific aspect, the water activity is calculated using Equations 12-15.

In one aspect, the method further comprises measuring the relative humidity. In a specific aspect, the method further comprises calculating the average relative humidity using Equation 16.

In one aspect, the time point is between 10 seconds and 168 hours, between 5 minutes and 168 hours, between 3 hours and 168 hours, between 6 hours and 240 hours, about 10 seconds, about 1 hour, about 6 hours, about 12 hours, about 24 hours, about 36 hours, about 48 hours, about 72 hours, about 96 hours, about 120 hours, about 144 hours, or about 168 hours, or about 240 hours. In one aspect, manual measurements may be taken as frequent as every five minutes to provide additional datapoints and the process may potentially be automated to continuously take measurements and make calculations in real-time.

In one aspect, the tubing is vinyl tubing, platinum-cured silicone tubing, peroxide cured silicone tubing, high-density polyethylene (HDPE) tubing, fluoropolymer tubing, thermoplastic elastomer tubing (TPE) or similar thermoplastic elastomer tubing material.

In one aspect, the method further comprises using the calculated change in concentration at a first time point to iteratively calculate change in concentration at subsequent time point at least once. In a specific aspect, the iterative calculation is calculated using Equations 4 and 6.

In one aspect, a temperature of the tubing is between about 5° C. and about 30° C., between about 15° C. and about 30° C., between about 15° C. and about 25° C., between about 15° C. and about 21° C., between about 16° C. and about 24° C., between about 17° C. and about 23° C., between about 18° C. and about 21° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C.

In one aspect, the change in concentration is due to water loss from diffusive mass transfer.

This disclosure additionally provides a method for selecting tubing for a sample including a protein. In some exemplary embodiments, the method comprises (a) obtaining a sample including a protein stored in at least two tubings; (b) using said sample and each of said tubings to generate models of water lost from said sample over time in each of said tubings; and (c) selecting a tubing on the basis of less predicted water loss in step (b).

In one aspect, the models are generated using equations 17, 18 and 20.

In one aspect, the sample is cell culture fluid (CCF), harvested cell culture fluid (HCCF), any step in the downstream processing of a drug substance (DS), drug substance, or a drug product (DP).

In one aspect, the tubing internal diameter is known. In a specific aspect, the tubing internal diameter (ID) is between about 0.1 mm and about 32 mm, between about 0.2 mm and about 26 mm, between about 0.5 mm and about 16 mm, between about 0.8 mm and about 13 mm, between about 0.8. mm and about 10 mm, about 0.1 mm, about 0.2 mm, about 0.3 mm, about 0.5 mm, about 0.6 mm, about 0.8 mm, about 1 mm, about 1.2 mm, about 1.3 mm, about 1.6 mm, about 2.4 mm, about 3.2 mm, about 4.8 mm, about 6.4 mm, about 8 mm, about 9.6 mm, about 12.7 mm, about 15.9 mm, about 19 mm, about 25.4 mm, or about 31.8 mm.

In one aspect, the tubing thickness is known. In a specific aspect, the tubing thickness is between about 0.1 mm and about 10 mm, between about 0.2 mm and about 8 mm, between about 0.5 mm and about 7 mm, about 0.25 mm, about 0.4 mm, about 0.5 mm, about 0.7 mm, about 0.8 mm, about 1.6 mm, about 1.8 mm, about 2.1 mm, about 2.4 mm, about 3.2 mm, about 4 mm, about 4.8 mm, about 6.4 mm, about 7 mm, about 8 mm, about 9 mm, or about 10 mm. The size of the tubing can have any combination of ID and thickness described above and corresponding tubing surface area-to-volume ratio.

In one aspect, the tubing surface area-to-volume ratio is known. In a specific aspect, the tubing surface area-to-volume ratio is between about 0.5 mm⁻¹ and about 5 mm⁻¹, about 0.5 mm⁻¹, about 0.83 mm⁻¹, about 1 mm⁻¹, about 1.25 mm⁻¹, about 1.26 mm⁻¹, about 1.5 mm⁻¹, about 1.54 mm⁻¹, about 1.57 mm⁻¹, about 1.67 mm⁻¹, about 2 mm⁻¹, about 2.11 mm⁻¹, about 2.5 mm⁻¹, about 2.52 mm⁻¹, about 3 mm⁻¹, about 3.1 mm⁻¹, about 3.15 mm⁻¹, about 3.33 mm⁻¹, about 3.5 mm⁻¹, about 4 mm⁻¹, about 4.12 mm⁻¹, about 4.5 mm⁻¹, about 5 mm⁻¹, about 6.25 mm⁻¹, about 6.67 mm⁻¹, about 8.0 mm⁻¹, about 13.33 mm⁻¹, about 20.0 mm⁻¹, or about 40.0 mm⁻¹.

In one aspect, the sample volume is known. In another aspect, the density is known. In another aspect, the concentration of excipients is known. In another aspect, the concentration of the protein is known. In a further aspect, the water activity of the sample is known. In a specific aspect, the water activity is calculated using Equations 12-15.

In one aspect, the method further comprises measuring the relative humidity. In a specific aspect, the method further comprises calculating the average relative humidity using Equation 16.

In one aspect, the time point is between 10 seconds and 168 hours, between 5 minutes and 168 hours, between 3 hours and 168 hours, between 6 hours and 240 hours, about 10 seconds, about 1 hour, about 6 hours, about 12 hours, about 24 hours, about 36 hours, about 48 hours, about 72 hours, about 96 hours, about 120 hours, about 144 hours, or about 168 hours, or about 240 hours. In one aspect, manual measurements may be taken as frequent as every five minutes to provide additional datapoints and the process may potentially be automated to continuously take measurements and make calculations in real-time.

In one aspect, at least one of the tubings vinyl tubing, platinum-cured silicone tubing, peroxide cured silicone tubing, high-density polyethylene (HDPE) tubing, fluoropolymer tubing, thermoplastic elastomer tubing (TPE) or similar thermoplastic elastomer tubing material.

In one aspect, the method further comprises using the calculated water loss at a first time point to iteratively calculate water loss at a later time point at least once. In a specific aspect, the iterative calculation is calculated using Equations 4 and 6.

In one aspect, a temperature of each of the tubing is between about 5° C. and about 30° C., between about 15° C. and about 30° C., between about 15° C. and about 25° C., between about 15° C. and about 21° C., between about 16° C. and about 24° C., between about 17° C. and about 23° C., between about 18° C. and about 21° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C.

In one aspect, the water loss is due to diffusive mass transfer.

This disclosure further provides a method for selecting a range of hold times for a sample in tubing. In some exemplary embodiments, the method comprises (a) obtaining a sample including a protein stored in tubing; (b) using said sample and said tubing to generate a model of change in concentration of said protein over time in said tubing; and (c) selecting a range of hold times on the basis of predicted change in concentration of said protein in step (b).

In one aspect, a range of hold times is selected to prevent a change in concentration of said protein beyond a determined threshold of percent concentration change. In a specific aspect, the determined threshold of percent concentration change is about 15%, about 10%, about 8%, about 5%, about 2%, or about 1%. In another specific aspect, the determined threshold of percent concentration change is about 10%.

In one aspect, the model is generated using equations 17, 18, 20, and 21.

In one aspect, the sample is cell culture fluid (CCF), harvested cell culture fluid (HCCF), any step in the downstream processing of a drug substance (DS), drug substance, or a drug product (DP).

In one aspect, the tubing internal diameter is known. In a specific aspect, the tubing internal diameter (ID) is between about 0.1 mm and about 32 mm, between about 0.2 mm and about 26 mm, between about 0.5 mm and about 16 mm, between about 0.8 mm and about 13 mm, between about 0.8 mm and about 10 mm, about 0.1 mm, about 0.2 mm, about 0.3 mm, about 0.5 mm, about 0.6 mm, about 0.8 mm, about 1 mm, about 1.2 mm, about 1.3 mm, about 1.6 mm, about 2.4 mm, about 3.2 mm, about 4.8 mm, about 6.4 mm, about 8 mm, about 9.6 mm, about 12.7 mm, about 15.9 mm, about 19 mm, about 25.4 mm, or about 31.8 mm.

In one aspect, the tubing thickness is known. In a specific aspect, the tubing thickness is between about 0.1 mm and about 10 mm, between about 0.2 mm and about 8 mm, between about 0.5 mm and about 7 mm, about 0.25 mm, about 0.4 mm, about 0.5 mm, about 0.7 mm, about 0.8 mm, about 1.6 mm, about 1.8 mm, about 2.1 mm, about 2.4 mm, about 3.2 mm, about 4 mm, about 4.8 mm, about 6.4 mm, about 7 mm, about 8 mm, about 9 mm, or about 10 mm. The size of the tubing can have any combination of ID and thickness described above and corresponding tubing surface area-to-volume ratio.

In one aspect, the tubing surface area-to-volume ratio is known. In a specific aspect, the tubing surface area-to-volume ratio is between about 0.5 mm⁻¹ and about 5 mm⁻¹, about 0.5 mm⁻¹, about 0.83 mm⁻¹, about 1 mm⁻¹, about 1.25 mm⁻¹, about 1.26 mm⁻¹, about 1.5 mm⁻¹, about 1.54 mm⁻¹, about 1.57 mm⁻¹, about 1.67 mm⁻¹, about 2 mm⁻¹, about 2.11 mm⁻¹, about 2.5 mm⁻¹, about 2.52 mm⁻¹, about 3 mm⁻¹, about 3.1 mm⁻¹, about 3.15 mm⁻¹, about 3.33 mm⁻¹, about 3.5 mm⁻¹, about 4 mm⁻¹, about 4.12 mm⁻¹, about 4.5 mm⁻¹, about 5 mm⁻¹, about 6.25 mm⁻¹, about 6.67 mm⁻¹, about 8.0 mm⁻¹, about 13.33 mm⁻¹, about 20.0 mm⁻¹, or about 40.0 mm⁻¹.

In one aspect, the sample volume, concentration of excipients or density is known

In one aspect, the water activity of said sample is known. In a specific aspect, the water activity is calculated using Equations 12-15.

In one aspect, the method further comprises measuring the relative humidity. In a specific aspect, the method further comprises calculating the average relative humidity using Equation 16.

In one aspect, the time point is between 10 seconds and 168 hours, between 5 minutes and 168 hours, between 3 hours and 168 hours, between 6 hours and 240 hours, about 10 seconds, about 1 hour, about 6 hours, about 12 hours, about 24 hours, about 36 hours, about 48 hours, about 72 hours, about 96 hours, about 120 hours, about 144 hours, or about 168 hours, or about 240 hours. In one aspect, manual measurements may be taken as frequent as every five minutes to provide additional datapoints and the process may potentially be automated to continuously take measurements and make calculations in real-time.

In one aspect, the tubing is vinyl tubing, platinum-cured silicone tubing, peroxide cured silicone tubing, high-density polyethylene (HDPE) tubing, fluoropolymer tubing, thermoplastic elastomer tubing (TPE) or similar thermoplastic elastomer tubing material.

In one aspect, the method further comprises using the calculated change in concentration at a first time point to iteratively calculate change in concentration at a later time point at least once. In a specific aspect, the iterative calculation is calculated using Equations 4 and 6.

The change in concentration is calculated from the change in tubing weight according to Equation 21.

In one aspect, a temperature of the tubing is between about 5° C. and about 30° C., between about 15° C. and about 30° C., between about 15° C. and about 25° C., between about 15° C. and about 21° C., between about 16° C. and about 24° C., between about 17° C. and about 23° C., between about 18° C. and about 21° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C.

In one aspect, the change in concentration is due to water loss from diffusive mass transfer.

These, and other, aspects of the present invention will be better appreciated and understood when considered in conjunction with the following description and accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1 shows an arrangement of tubings in a biosafety cabinet under ambient conditions, according to an exemplary embodiment.

FIG. 2A shows a change in tubing weight over time for tubing A, according to an exemplary embodiment.

FIG. 2B shows a change in tubing weight over time for tubing B, according to an exemplary embodiment.

FIG. 2C shows a change in tubing weight over time for tubing C, according to an exemplary embodiment.

FIG. 3A shows a change in concentration of mAb A over time with a starting concentration of 50 mg/mL in tubings A, B, and C, as measured by reversed phase ultra performance liquid chromatography (RP-UPLC), according to an exemplary embodiment.

FIG. 3B shows a change in concentration of mAb A over time with a starting concentration of 150 mg/mL in tubings A, B, and C, as measured by reversed phase ultra performance liquid chromatography (RP-UPLC), according to an exemplary embodiment.

FIG. 4 shows a change in tubing weight (%) plotted against a change in protein concentration (%) for mAb A formulations of starting concentration 50 mg/mL and 150 mg/mL filled in tubings A, B, and C, according to an exemplary embodiment.

FIG. 5 shows a change in tubing weight over time in tubing A based on drug substance in the tubing, according to an exemplary embodiment.

FIG. 6 shows a change in tubing weight over time in tubing A based on water activity in the tubing using NaCl solutions, according to an exemplary embodiment.

FIG. 7 shows a change in tubing weight over time in tubing A based on water activity in the tubing using CaCl₂) solutions, according to an exemplary embodiment.

FIG. 8 shows a correlation between rate of change of tubing weight and water activity in the tubing for various protein and salt solutions, water, and buffer in tubing A, according to an exemplary embodiment.

FIG. 9 shows a correlation between effective diffusivity and water activity for various formulation-tubing combinations, according to an exemplary embodiment.

FIG. 10A shows a predicted and experimentally obtained change in tubing weight as a function of time for 0.5 mL 50 mg/mL mAb A DS in 0.8 mm ID tubing, according to an exemplary embodiment.

FIG. 10B shows a predicted and experimentally obtained change in tubing weight as a function of time for 3 mL 50 mg/mL mAb A DS in 1.2 mm ID tubing, according to an exemplary embodiment.

FIG. 10C shows a predicted and experimentally obtained change in tubing weight as a function of time for 3 mL 50 mg/mL mAb A DS in 3.2 mm ID tubing, according to an exemplary embodiment.

FIG. 10D shows a predicted and experimentally obtained change in tubing weight as a function of time for 0.5 mL 150 mg/mL mAb A DS in 0.8 mm ID tubing, according to an exemplary embodiment.

FIG. 10E shows a predicted and experimentally obtained change in tubing weight as a function of time for 3 mL 150 mg/mL mAb A DS in 1.2 mm ID tubing, according to an exemplary embodiment.

FIG. 10F shows a predicted and experimentally obtained change in tubing weight as a function of time for 3 mL 150 mg/mL mAb A DS in 3.2 mm ID tubing, according to an exemplary embodiment.

FIG. 10G shows a predicted and experimentally obtained change in tubing weight as a function of time for 0.5 mL 175 mg/mL mAb A DS in 0.8 mm ID tubing, according to an exemplary embodiment.

FIG. 10H shows a predicted and experimentally obtained change in tubing weight as a function of time for 3 mL 175 mg/mL mAb A DS in 1.2 mm ID tubing, according to an exemplary embodiment.

FIG. 10I shows a predicted and experimentally obtained change in tubing weight as a function of time for 3 mL 175 mg/mL mAb A DS in 3.2 mm ID tubing, according to an exemplary embodiment.

FIG. 11 shows an average change in tubing weight as a function of time for C-flex tubing (3.2 mm ID, 1.6 mm thickness) containing 150 mg/mL mAb A DS compared to a model-predicted change in weight for platinum-cured silicone tubing (3.2 mm ID, 1.6 mm thickness) containing 150 mg/mL mAb A DS, according to an exemplary embodiment.

### DETAILED DESCRIPTION

Drug substance (DS) encounters a variety of materials (metal, elastomers, single use disposable systems, etc.) during drug product (DP) manufacturing or fill/finish operation, and it is important to evaluate physicochemical compatibility of DS with these contact parts. Silicone tubing is one such elastomeric component used during DP manufacturing to facilitate fluid transfer and filling operation (Colas et al., “Silicone tubing for pharmaceutical processing,” DuPont, accessed February 2022).

Silicone tubing has known permeability to various low molecular weight species and gases (Colas et al.; Saller et al., 2017, Eur J Pharm Biopharm, 112:109-118; Eisner et al., 2019, PDA Journal of Pharmaceutical Science and Technology, 73(5):443-458). The permeability of silicone tubing to gases and organic compounds has been quantified in literature (Zhang et al., 2006, SAMPE Fall Technical Conference, proceedings, Coatings and Sealants Section, Nov. 6-9, 2006, Dallas, TX; “Permeability of Catheter and Tubing Materials,” Instech Blog, accessed January 2022). The permeability of tubing materials used in catheters for animal studies to a solution of dye in water has also been studied qualitatively and it has been concluded that water loss occurs from silicone tubing (Instech Blog). However, the potential impact of water loss from silicone tubing on drug and/or excipient concentration in the context of DP manufacturing has not been quantified or mentioned widely in literature.

During process characterization and development studies to support tech transfer of a monoclonal antibody (mAb) DP, an increase in protein concentration over time while the material was held in contact with silicone tubing was observed. Therefore, a study was designed to develop a better understanding of this phenomenon and the factors involved. Silicone tubings with different internal diameters and wall thicknesses were evaluated for various mAb formulations and protein concentrations. It was hypothesized that an increase in protein concentration would occur because of water permeability through silicone tubing, and the rate of water loss during static hold of DS inside the tubing would be a function of tubing parameters, specifically the tubing internal diameter and wall thickness.

Initially, tubing weight loss was monitored for water and DS dilution buffer (also referred to as “buffer” in subsequent text) as controls, and formulations of one type of mAb as test. In addition to weight loss, protein concentration in the tubings over time was also monitored. Both tubing weight change and protein concentration change were monitored as a function of tubing parameters across three different tubing types. A relationship was developed between the change in protein concentration and the change in tubing weight. Subsequently, the weight loss in additional mAb formulations held in silicone tubing types was monitored to determine the effects on the rate of weight loss of (1) mAb candidates, (2) the initial protein concentration; and (3) formulation composition. Next, the effect of formulation water activity on the rate of water loss was investigated using NaCl and CaCl₂ salt solutions of varying concentrations. Finally, the mechanism of weight loss in the silicone tubings was investigated through diffusion-based modeling. Effective diffusion coefficients based on Fick's first law of diffusion were calculated for the tubing sets. A diffusion-based water loss mechanistic model incorporating tubing dimensions and formulation characteristics was developed based on the collected data to predict the amount of weight loss over long hold times for a given tubing-formulation combination. The model was verified by monitoring the water loss from mAb-filled tubings of dimensions different from those used in model development. The efforts helped to facilitate a robust process characterization for DP tech transfer with the goal of designing more representative process development studies.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one.” As used herein, the terms “include,” “includes,” and “including” are meant to be non-limiting and are understood to mean “comprise,” “comprises,” and “comprising” respectively. The terms “about” and “approximately” should be understood to permit standard variation of ±5%, and where ranges are provided, endpoints are included

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated by reference). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of. IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the present invention, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or KX-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

As used herein, a “protein pharmaceutical product,” “biopharmaceutical product” or “biotherapeutic” includes an active ingredient which can be fully or partially biological in nature. In one aspect, the protein pharmaceutical product can comprise a peptide, a protein, a fusion protein, an antibody, an antigen, vaccine, a peptide-drug conjugate, an antibody-drug conjugate, a protein-drug conjugate, cells, tissues, or combinations thereof. In another aspect, the protein pharmaceutical product can comprise a recombinant, engineered, modified, mutated, or truncated version of a peptide, a protein, a fusion protein, an antibody, an antigen, vaccine, a peptide-drug conjugate, an antibody-drug conjugate, a protein-drug conjugate, cells, tissues, or combinations thereof.

As used herein, a “sample” can be obtained from any step of a bioprocess, such as cell culture fluid (CCF), harvested cell culture fluid (HCCF), any step in the downstream processing, drug substance (DS), or a drug product (DP) comprising the final formulated product. In some specific exemplary embodiments, the sample can be selected from any step of the downstream process of clarification, chromatographic production, or filtration.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reverse phase liquid chromatography, ion-exchange chromatography, size exclusion chromatography, affinity chromatography, hydrophobic interaction chromatography, hydrophilic interaction chromatography, or mixed-mode chromatography. In some aspects, a sample containing the at least one protein of interest or peptide digest can be subjected to any one of the aforementioned chromatographic methods or a combination thereof. Analytes separated using chromatography will feature distinctive retention times, reflecting the speed at which an analyte moves through the chromatographic column. Analytes may be compared using a chromatogram, which plots retention time on one axis and measured signal on another axis, where the measured signal may be produced from, for example, UV detection or fluorescence detection.

This disclosure provides a method for predicting water loss from a sample, for predicting protein concentration change, for selecting a range of hold times for a sample including a protein in tubing, and for selecting tubing based on predicted water loss, using, for example, Equations 1-21 as set forth in this disclosure. It should be understood that the embodiments and aspects described in this disclosure may pertain to any of these described methods.

In some exemplary embodiments, the sample is water, buffer, cell culture fluid (CCF), harvested cell culture fluid (HCCF), any step in the downstream processing of a drug substance (DS), drug substance, or a drug product (DP). In some specific embodiments, the drug product includes a therapeutic protein. In further specific embodiments, the therapeutic protein is an antibody, a monoclonal antibody, a bispecific antibody, a fusion protein, a receptor, an antibody-drug conjugate, or an antibody fragment.

In some exemplary embodiments, the tubing internal diameter is known. In some exemplary embodiments, a tubing internal diameter is between about 0.1 mm and about 32 mm, between about 0.2 mm and about 26 mm, between about 0.5 mm and about 16 mm, between about 0.8 mm and about 13 mm, about 0.8 mm and about 10 mm, about 0.1 mm, about 0.2 mm, about 0.3 mm, about 0.5 mm, about 0.6 mm, about 0.8 mm, about 1 mm, about 1.2 mm, about 1.3 mm, about 1.6 mm, about 2.4 mm, about 3.2 mm, about 4.8 mm, about 6.4 mm, about 8 mm, about 9.6 mm, about 12.7 mm, about 15.9 mm, about 19 mm, about 25.4 mm, or about 31.8 mm.

In some exemplary embodiments, the tubing thickness is known. In some exemplary embodiments, the tubing thickness is between about 0.1 mm and about 10 mm, between about 0.2 mm and about 8 mm, between about 0.5 mm and about 7 mm, about 0.25 mm, about 0.4 mm, about 0.5 mm, about 0.7 mm, about 0.8 mm, about 1.6 mm, about 1.6 mm, about 2.1 mm, about 2.4 mm, about 3.2 mm, about 4 mm, about 4.8 mm, about 6.4 mm, about 7 mm, about 8 mm, about 9 mm, or about 10 mm. The size of the tubing can have any combination of ID and thickness described above and corresponding tubing surface area-to-volume ratio.

In one aspect, the tubing surface area-to-volume ratio is known. In a specific aspect, the tubing surface area-to-volume ratio is between about 0.5 mm⁻¹ and about 5 mm⁻¹, about 0.5 mm⁻¹, about 0.83 mm⁻¹, about 1 mm⁻¹, about 1.25 mm⁻¹, about 1.26 mm⁻¹, about 1.5 mm⁻¹, about 1.54 mm⁻¹, about 1.57 mm⁻¹, about 1.67 mm⁻¹, about 2 mm⁻¹, about 2.11 mm⁻¹, about 2.5 mm⁻¹, about 2.52 mm⁻¹, about 3 mm⁻¹, about 3.1 mm⁻¹, about 3.15 mm⁻¹, about 3.33 mm⁻¹, about 3.5 mm⁻¹, about 4 mm⁻¹, about 4.12 mm⁻¹, about 4.5 mm⁻¹, about 5 mm⁻¹, about 6.25 mm⁻¹, about 6.67 mm⁻¹, about 8.0 mm⁻¹, about 13.33 mm⁻¹, about 20.0 mm⁻¹, or about 40.0 mm⁻¹.

In some exemplary embodiments, the sample water activity is known. In some exemplary embodiments, the sample water activity is between about 0.7 and about 1, between about 0.8 and about 1, between about 0.85 and about 1, between about 0.9 and about 1, between about 0.95 and about 1, between about 0.95 and about 0.99, about 0.7, about 0.71, about 0.72, about 0.73, about 0.74, about 0.75, about 0.76, about 0.77, about 0.78, about 0.79, about 0.80, about 0.81, about 0.82, about 0.83, about 0.84, about 0.85, about 0.86, about 0.87, about 0.88, about 0.89, about 0.90, about 0.91, about 0.92, about 0.93, about 0.94, about 0.95, about 0.96, about 0.97, about 0.98, about 0.985, about 0.99, about 0.995, or about 1.

In some exemplary embodiments, the method further comprises measuring the relative humidity. In some exemplary embodiments, the relative humidity is between about 35% and about 65%, between about 40% and about 50%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, or about 65%.

In some exemplary embodiments, the time point is between 10 seconds and 168 hours, between 5 minutes and 168 hours, between 3 hours and 168 hours, between 6 hours and 240 hours, about 10 seconds, about 1 hour, about 6 hours, about 12 hours, about 24 hours, about 36 hours, about 48 hours, about 72 hours, about 96 hours, about 120 hours, about 144 hours, or about 168 hours, or about 240 hours. In one aspect, manual measurements may be taken as frequent as every five minutes to provide additional datapoints and the process may potentially be automated to continuously take measurements and make calculations in real-time.

In some exemplary embodiments, the tubing is platinum-cured silicone tubing. Exemplary tubing includes, for example, Masterflex tubing with the following catalog numbers: 96410-14, 96410-15, 96410-25; C-Flex® tubing from Saint-Gobain (374-125-2); Flexicon Accusil™ silicone tubing from Watson-Marlow Limited; and any other tubing that may be of use in storing, transporting or processing a sample. It should be understood that the present invention is not limited to the aforementioned tubing. The principles of the present invention, namely water loss due to diffusive mass transfer through polymeric tubing, may apply to any potentially porous tubing material, and therefore the method of the present invention is not limited to tubing made of silicone. Similarly, the same principles of water loss due to diffusive mass transfer through a polymeric surface apply to any aqueous sample and are not limited to a sample including a protein or a drug product.

A prediction of a change in concentration of a protein at a time point, or at several time points, may be used to determine a range of acceptable hold times for a sample in tubing. A threshold may be determined as the maximum percent change in protein concentration that is acceptable for a given process or at a particular step of the process, and a range of hold times may be determined for which the predicted change in protein concentration is below said threshold. In some exemplary embodiments, a threshold of a maximum acceptable percent change in protein concentration during a hold time is plus or minus about 15%, about 14%, about 13%, about 12%, about 11%, about 10%, about 9%, about 8%, about 7%, about 6%, about 5%, about 4%, about 3%, about 2%, about 1%, or about 0.1%. In a specific embodiment, a threshold of a maximum acceptable percent change in protein concentration during a hold time is plus or minus about 10%.

It is understood that the present invention is not limited to any of the aforesaid protein(s), protein(s) of interest, antibody(s), sample(s), sample volume(s), tubing(s), chromatographic method(s), database(s), bioinformatics tool(s), pH, temperature(s), or concentration(s), and any protein(s), protein(s) of interest, antibody(s), sample(s), sample volume(s), tubing(s), chromatographic method(s), database(s), bioinformatics tool(s), pH, temperature(s), or concentration(s) can be selected by any suitable means.

The present invention will be more fully understood by reference to the following examples. They should not, however, be construed as limiting the scope of the invention.

### EXAMPLES

Materials. Materials used include water for injection (WFI) (USP sterile grade, Intermountain Life Sciences); L-histidine (USP grade, Ph. Eur. Grade, Ajinomoto U.S.A. Inc.); L-histidine monohydrochloride monohydrate (Ph. Eur. Grade, Ajinomoto U.S.A. Inc.); L-proline (USP grade, Ph. Eur. Grade, Ajinomoto U.S.A. Inc.); L-arginine hydrochloride (USP grade, Ph. Eur. Grade, Avantor Performance Chemicals); polysorbate 80 super refined (10% w/w, procured in-house); mAb ADS (150 mg/mL, Regeneron Pharmaceuticals); mAb B DS (120 mg/mL, Regeneron Pharmaceuticals); mAb C DS (100 mg/mL, Regeneron Pharmaceuticals); mAb D DS (175 mg/mL, Regeneron Pharmaceuticals); trifluoroacetic acid (TFA, Honeywell, Biosynthesis, HPLC grade); acetonitrile (UV grade, Honeywell); sodium phosphate monobasic (ACS grade, J.T. Baker); sodium phosphate dibasic, 7-hydrate, crystal (ACS grade, J.T. Baker); sodium chloride (ACS grade, J.T. Baker); calcium chloride (ACS grade, Spectrum Chemical); and ethanol (200 proof, Koptec). It should be noted that the formulation composition varied between mAbs A-D. The composition used in the study are shown in Table 1.

TABLE 1
Formulation composition for mAb A-D DS used in the study.

| mAb A DS | mAb B DS | mAb C DS | mAb D DS |
|---|---|---|---|
| mAb A, 150 mg/mL | mAb B, 120 mg/mL | mAb C, 100 mg/mL | mAb D, 175 mg/mL |
| 10 mM histidine, | 10 mM L-histidine, | 10 mM histidine, | 21 mM histidine, |
| pH 6.0 ± 0.2 | pH 6.0 ± 0.2 | pH 6.0 ± 0.2 | pH 6.0 ± 0.2 |
| 3% L-proline (w/v) | 8% sucrose (w/v) | 10% sucrose (w/v) | 5% sucrose (w/v) |
| 0.1% polysorbate | 0.1% polysorbate | 0.1% polysorbate | 0.2% polysorbate |
| 80 (w/v) | 80 (w/v) | 80 (w/v) | 20 (w/v) |
| 70 mM Arginine-HCl | | | 45 mM Arginine-HCl |

Platinum-cured silicone peristaltic pump tubing (Masterflex) with the following catalog numbers was used: 96410-14 (Tubing A), 96410-15 (Tubing B), 96410-25 (Tubing C), and C-Flex® tubing was used from Saint-Gobain (374-125-2). Flexicon Accusil™ silicone tubing (part numbers: 84-103-012, 84-103-008, 84-103-032) from Watson-Marlow Limited was used. The tubing was cut into pieces of length sufficient to hold 3 mL liquid that were autoclaved at 250° F. for 20 minutes and subsequently dried for 30 minutes before use. The Flexicon 0.8 mm ID tubing (part number: 84-103-008) was cut into pieces sufficient to hold 0.5 mL to ease handling of tubing length. All solutions prepared were filtered through a sterile Stericup® Quick Release Durapore® 0.22 μm polyvinylidene fluoride (PVDF) filter unit (Millipore) before use.

Instrumentation. A Mettler Toledo XSE205 DualRange balance was used for weighing the tubings to measure potential water loss. Three weights were recorded for each tubing at each collection time point, and the average value was used for further analysis.

Reverse phase-ultra performance liquid chromatography (RP-UPLC) analysis was performed using a Waters Acquity UPLC system with an Agilent Technologies Zorbax 300 SB-CN column and a tunable UV detector to measure protein concentration. Two mobile phases were prepared for RP-UPLC, consisting of 0.1% TFA in water (solvent A) and 0.1% TFA in acetonitrile (solvent B). A gradient method was used starting with 90% of solvent A and 10% of solvent B for 1 minute, followed by 10% of Solvent A and 90% of Solvent B for 5.4 minutes, followed by 90% of solvent A and 10% of solvent B for 9 minutes. The total flow rate was maintained at 1 mL/min with a run time of 5 minutes. The column temperature was maintained at 60° C. and the detection wavelength at 280 nm. Three injections were performed on each sample for accuracy.

A Vapro® 5600 Vapor Pressure Osmometer was used to record osmolalities of mAb A formulations and buffer. Measurements were performed in triplicate for accuracy. A TES 1340 hot-wire anemometer was used to record air velocity data. Relative humidity data were obtained by the Facilities Management department in Regeneron and using a VWR Traceable® hygrometer (Cat. No. 35519-050) hygrometer.

Data analysis. JMP® statistical software was used to perform the required statistical analysis. The weight change data obtained from the study was fitted to Fick's first law of diffusion (Bird et al., 1960, “Transport Phenomena,” John Wiley and Sons, Inc.) to obtain the effective diffusivity at each time point, as shown in Equation 2. The following calculations were involved in diffusion-based modeling. Fick's 1^{st} law shown in Equation 1 was used as the basis:

$${J}_{{water}_{Δ t}} ( \frac{g}{{m}^{2} s} ) = - D ( t ) \frac{Δ {C}_{water} ( t ) ( \frac{g}{{m}^{3}} )}{Δ x ( m )} \qquad ( 1 )$$

Equation 1 can be re-arranged to give Equation 2:

$$D ( t ) ( \frac{{m}^{2}}{s} ) = \frac{- {J}_{{water}_{Δ t}} ( \frac{g}{{m}^{2} s} ) * Δ x ( m )}{Δ {C}_{water} ( t ) ( \frac{g}{{m}^{3}} )} \qquad ( 2 )$$

J_{water}_{_{Δt}} in Equation 2 is derived using Equations 3-6:

$${J}_{{water}_{Δ t}} = \frac{Δ Weight ( t )}{SA ( t ) * Δ t} \qquad ( 3 )$$

$$Δ Weight ( t ) = Tubing {weight}_{t} - Tubing {weight}_{t + Δ t} \qquad ( 4 )$$

$$SA ( t ) = \frac{4 V ( t )}{ID} \qquad ( 5 )$$

$$V ( t ) = V ( {t}_{0} ) - Δ Volume of water ( t ) = V ( {t}_{0} ) - Δ Weight ( t ) \qquad ( 6 )$$

The concentration gradient of water (ΔC_{water}(t)) in Equation 2 is derived using Equations 7-10:

ΔC_{water}=C_{water}_{_{tubing}}(t)−C_{water}_{_{ambient}}(t) (7)

C_{water}_{_{tubing}}(t) and C_{water}_{_{ambient}}(t) can be determined from the corresponding water activities as shown in equations 8a and 8b (Rhodes, G. “Concentration and activity.” (2000) Biochemical Goodies Blog https://www.bgu.ac.il/˜aflaloc/BioHTML/Goodies/ConcAct.html).

C_{water}_{_{tubing}}(t)=aw_{tubing}(t)*C_{water}_{ₚᵤᵣₑ} (8a)

C_{water}_{_{ambient}}(t)=aw_{ambient}(t)*C_{waterpure} (8b)

Where, C_{water}_{_{tubing}}(t) and C_{water}_{_{ambient}}(t) are the actual water concentration in the tubing and actual ambient water concentration respectively and C_{water}_{ₚᵤᵣₑ} is the standard concentration of pure water i.e. 55.5 M.

Substituting equations 8a and 8b in equation 7 would result in equations 9a and 9b.

$$Δ {C}_{water} = {aw}_{tubing} ( t ) * {C}_{{water}_{pure}} - {aw}_{ambient} ( t ) * {C}_{{water}_{pure}} \qquad ( 9 a )$$

$$Δ {C}_{water} = {C}_{{water}_{pure}} * [ {aw}_{tubing} ( t ) - {aw}_{ambient} ( t ) ] \qquad ( 9 b )$$

$${C}_{{water}_{pure}} = \frac{55.5 * 18}{1 {0}^{- 3}} \frac{g}{{m}^{3}} \approx 1000000 \frac{g}{{m}^{3}} \qquad ( 10 )$$

Overall, substituting Equations 7-10 in Equation 2 results in Equation 11:

$$D ( t ) ( \frac{{m}^{2}}{s} ) = \frac{{J}_{{water}_{Δ t}} ( \frac{g}{{m}^{2} s} ) * Δ x ( m )}{{C}_{{water}_{pure}} * [ {aw}_{tubing} ( t ) - {aw}_{ambient} ( t ) ] ( \frac{g}{{m}^{3}} )} \qquad ( 11 )$$

Equations 12-15 describe the calculation of the water activity at time t (aw(t)) in Equation 8. Equation 12 is used to calculate the molarity of protein and excipients at time t (C_{protein+excipients}(t)). Equation 13 uses C_{protein+excipients}(t) to calculate the solution osmolality at time t. The solution osmolality is converted to the freezing point depression (T_{f}−T (t)) in Equation 14 that is used as an input in the Hildebrand-Scott equation (Equation 15) (Miyawaki et. al. “Activity and Activity Coefficient of Water in Aqueous Solutions and Their Relationships with Solution Structure Parameters” (1997). Biosci. Biotech. Biochem., 61 (3), 466-469) to calculate aw_{tubing}(t).

$${C}_{protein + excipients} ( t ) = \frac{{C}_{protein + excipients} ( {t}_{0} ) * V ( {t}_{0} )}{V ( t )} \qquad ( 12 )$$

$$Osmolality = \frac{{C}_{protein + excipients} ( t ) * Osmolality ( {t}_{0} )}{{C}_{protein + excipients} ( {t}_{0} )} \qquad ( 13 )$$

$${T}_{f} - T ( t ) = Osmolality ( t ) ( \frac{Osm}{kg} ) * 1.86 \qquad ( 14 )$$

$$ln aw ( t ) = - \frac{Δ {H}_{f}}{R} * \frac{{T}_{f} - T ( t )}{{T}_{f} T ( t )} + \frac{Δ {C}_{f}}{R} * [ \frac{{T}_{f} - T ( t )}{T ( t )} - ln ( \frac{{T}_{f}}{T ( t )} ) ] \qquad ( 15 )$$

Equation 16 describes the calculation of the ambient water activity (aw_{ambient}(t)) using the relative humidity in the atmosphere. The relative humidity is averaged over the time interval Δt.

$${aw}_{ambient} ( t ) = \frac{[ Relative humidity ( t ) + Relative humdity ( t + Δ t ) ] ( \% )}{2 * 100} \qquad ( 16 )$$

t=Time point during the study, 0<t≤96 h (h);
D (t)=Effective diffusivity coefficient at time t (m²/s);
C_{water}_{_{tubing}}(t)=Concentration of water inside the tubing at time t (g/m³);
C_{water}_{_{ambient}}(t)=Concentration of water in the atmosphere at time t (g/m³);
Δx=Tubing wall thickness (m);
ΔWeight(t)=Incremental change in tubing weight between t and (t+Δt) h (g);
J_{water}_{_{Δt}}=Incremental flux of water between t and (t+Δt) h (g/m² s);
Δt=Time period from tᵢ to tⱼ h (s), where j>i≥0. For initial effective diffusivity coefficient calculations from experimental data, Δt=24 hours;
SA (t)=Surface area of tubing in contact with solution at time t (m²);
t₀=Time of study set-up (h);
V(t)=Solution volume in tubing at time t (m³);
ID=Tubing internal diameter (m);
C_{water}_{ₚᵤᵣₑ}=Concentration of pure water (55.5 M or 55555.55 mol/m³≈1000000 g/m³);
aw_{tubing}(t)=Water activity in solution in tubing at time t;
aw_{ambient}(t)=Water activity in the ambient atmosphere at time t;
C_{protein+excipients}(t)=Concentration of protein and excipients in solution at time t (g/m³);
T_{f}−T (t)=Freezing point depression at time t, where T is freezing point of solution and T_{f} is freezing point of water (K);
ΔH_{f}=Latent heat of water=6008 J/K/mol¹
ΔC_{f}=Change of specific heat of water=38.7 J/mol/K¹
R=Gas constant=8.314 J/K/mol

The following assumptions were made in these calculations: (1) The weight of water lost is equal to the volume of solution lost in the tubing, as the density of water is 1 g/mL

(2) For protein/buffer solutions, the concentration of protein and excipients is calculated using the dilution law assuming the total weight of protein and excipients remains constant as water is the only component diffusing out (for salt solutions, the dilution law is used assuming salt is the only excipient and that its weight remains constant with time). (3) For protein/buffer solutions, the initial osmolality is experimentally known and the osmolality of solution at a subsequent time is directly proportional to the concentration of protein and excipients present in the solution at that time. (4) The average relative humidity for a given time interval can be used to estimate ambient water activity instead of instantaneous relative humidity at each time point and the average relative humidity is calculated as the average of reported relative humidity at the two ends of the time interval.

The effective diffusivity obtained at each time point for the tubing-formulation combinations was used in further mechanistic modeling. The effective diffusivity values obtained using Equation 11 at each study time point were averaged across all time points between 0 to 96 hours to obtain an overall effective diffusivity for each formulation and tubing type

mAb A buffer preparation. A buffer was prepared as a control and for subsequent dilution of mAb A DS. The buffer consisted of 10 mM histidine, 70 mM arginine-HCl, 3% w/w proline, and 0.1% w/w polysorbate 80 added to WFI and a pH of 6.0±0.2. It was filtered through a 0.22 μm PVDF filter after preparation.

mAb A formulation preparation. A diluted formulation of mAb A of the desired concentration (50 mg/mL) was prepared from the as-obtained mAb ADS (150 mg/mL). The DS in required volume was mixed with buffer of the required volume using sterile pipettes. The formulation was filtered through a 0.22 μm PVDF filter before use.

Salt solutions preparation. NaCl and CaCl₂ solutions of various concentrations were prepared in MilliQ water on a weight-by-weight basis. Complete dissolution of salt was ensured. Concentrated CaCl₂ solutions were exothermic after preparation and were completely cooled before use. Table 7 lists the concentrations of the NaCl and CaCl₂ solutions prepared.

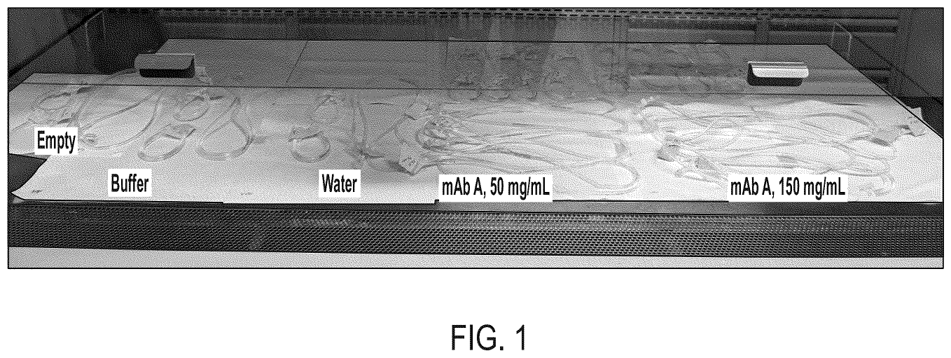

Study set-up. Initially, five tubing sets were prepared: water-filled, buffer-filled, and empty tubing sets as controls; mAb A 50 mg/mL-filled set; and mAb A 150 mg/mL-filled set. Each tubing set consisted of the three tubing types, A, B, and C. One tubing of each type A, B, and C was prepared for the water, buffer, along with an empty tubing. Four tubings of each type A, B, and C corresponding to four collection time points were prepared for each of the two formulations. Each tubing piece, excluding tubing in the empty set, was filled with 3 mL of solution using a syringe and the ends of the tubing were sealed using zip-ties. The tubings were placed in a biosafety cabinet under ambient temperature (about 22° C.) throughout the study. The air circulation in the biosafety cabinet was left off during the study. Initially, the weights of all tubings were recorded at time 0 hours. Subsequently, weights were recorded at four pre-determined time intervals: 24 h, 48 h, 72 h, and 96 h for mAb A 50 mg/mL and mAb A 150 mg/mL-filled tubings. The solutions were collected from the tubings and stored in cryo-tubes at 5° C. until further analysis for protein concentration by RP-UPLC. Water, buffer, and empty tubing weights were recorded at each of the time points after which the tubings were returned to the biosafety cabinet. The setup of the study for water, buffer, mAb A 150 mg/mL, and mAb A 50 mg/mL formulations in tubings is shown in FIG. 1Error! Reference source not found. One tubing piece of each type was used for the water and buffer control sets, while four tubing pieces of each type corresponding to the four time points were used for the formulation sets. Three weights were recorded per tubing.

For all formulation-tubing combinations used in subsequent experiments (mAbs B-D in tubings filled in 0.8 mm ID, 1.2 mm ID, 3.2 mm ID, or Tubing A), one tubing piece was filled with either 3 mL of formulation (for 1.2 mm ID, 3.2 mm ID, or Tubing A) or 0.5 mL formulation (for 0.8 mm ID). The weight of this tubing was monitored in the same manner in triplicate at four time points over 96 hours as for the mAb A, water, and buffer formulations without collecting any sample from the tubing for further analysis. The tubing was returned to the biosafety cabinet after each weight measurement. The air circulation in the biosafety cabinet was turned off for all studies. Manual measurements may have been taken as frequent as every five minutes to provide additional datapoints and the process could have been potentially automated to continuously take measurements and make calculations in real-time.

Throughout the study, the temperature in the biosafety cabinet was around 22° C. and the air velocity was measured to be around 0.000-0.002 m/s in the vicinity of the tubings with the airflow turned off.

### Example 1. Change in Tubing Weight Over Time

Initially, the water loss from silicone tubings filled with controls (water, buffer) and mAb A DS (50 mg/mL and 150 mg/mL) was investigated. Based on the previous literature, it was expected that water loss may occur from silicone tubing over time because of the permeable nature of the tubing (Instech Blog). Mass transfer of water from inside to outside of the tubing is expected to be dependent on the water activity difference, surface-area-to-volume ratio (SA/V) of the tubing, and the resistance to molecular diffusion. The rate of mass transfer in the tubings is expected to increase with an increase in SA/V and decrease in resistance to diffusion offered by the tubing wall thickness. The physical parameters associated with each tubing type used in the study are listed in Table 2. Table 2 describes the physical parameters of the tubing used in the study involving water, buffer, and mAb A formulations, including tubing internal diameter (ID), wall thickness, and surface area-to-volume ratio (SA/V), which can be calculated based on the tubing ID. All tubing was platinum-cured silicone peristaltic pump tubing purchased from MasterFlex. A constant fill volume of 3 mL was used for all tubing. Tubings A and C have the same wall thickness while tubings B and C have the same SA/V. It was hypothesized that the rate of mass transfer would be highest for tubing A with the highest SA/V, followed by tubing C with smaller thickness, and least for tubing B.

TABLE 2
Tubing parameters

| | | | Wall | Surface area- |
|---|---|---|---|---|
| Tubing catalog | Tubing | Tubing | thickness | to-volume ratio |
| number | reference | ID (mm) | (mm) | (SA/V) (mm⁻¹) |
| Masterflex 96410-14 | A | 1.6 | 1.6 | 2.5 |
| Masterflex 96410-15 | B | 4.8 | 2.4 | 0.83 |
| Masterflex 96410-25 | C | 4.8 | 1.6 | 0.83 |

The rate of mass transfer or water loss can be estimated by the change in tubing weight over time. Hence, the weights of each tubing set were analyzed as a function of time. The weights recorded as a function of time for the water, buffer, mAb A 150 mg/mL DS, and mAb A 50 mg/mL DS-filled tubing sets are listed in Table 3 and Table 4. Weight of empty tubing was also monitored over time as a negative control. It was found that the difference between the initial weight and weight at various time points of empty tubing (types, A, B and C) is less than 0.1%. For the other tubing sets and types, a decrease in weight was observed over time.

For Table 3, weights of tubing sets for water, buffer, and empty tubing were recorded from time 0 h after set-up to 96 h. Three tubing types (A, B, and C) were used in the study. One tubing of each type was used per set. Weights were recorded at 24 hours, 48 hours, 72 hours, and 96 hours after study set-up. Three weights were recorded per tubing and the average and standard deviation are reported. The tubings were placed in a biosafety cabinet under ambient temperature (˜22° C.) throughout the study. Relative humidity data was internally recorded by the Facilities Management department in Regeneron.

For Table 4, weights of tubing sets for formulations mAb A 50 mg/mL DS and mAb A 150 mg/mL DS were recorded from time 0 h after set-up to 96 h. Three tubing types (A, B, and C) were used in the study. One tubing of each type was dedicated to each time point, resulting in four tubings of each type for 24 hours, 48 hours, 72 hours, and 96 hours. Initial weight was recorded for each tubing at the beginning of the study. Weight was also recorded at the specified time point for each tubing after study set-up, for example, weight for the 72-hour-dedicated tubing was only recorded at study setup and at 72 hours. Three weights were recorded per tubing and the average and standard deviation are reported. The tubings were placed in a biosafety cabinet under ambient temperature (˜22° C.) throughout the study. Relative humidity data was internally recorded by the Facilities Management department in Regeneron.

TABLE 3
Weights of tubing sets for water, buffer, and empty tubing

| Tubing | Tubing | Weights (g) (average + SD) | | | | |
|---|---|---|---|---|---|---|
| Set | type | 0 h | 24 h | 48 h | 72 h | 96 h |
| Water | A | 35.326 ± | 35.156 ± | 34.959 ± | 34.783 ± | 34.625 ± |
| | | 0.000 | 0.000 | 0.001 | 0.000 | 0.000 |
| | B | 21.139 ± | 21.088 ± | 21.037 ± | 20.989 ± | 20.945 ± |
| | | 0.000 | 0.000 | 0.000 | 0.000 | 0.000 |
| | C | 14.556 ± | 14.498 ± | 14.431 ± | 14.368 ± | 14.307 ± |
| | | 0.000 | 0.000 | 0.000 | 0.000 | 0.000 |
| Buffer | A | 35.140 ± | 34.967 ± | 34.770 ± | 34.593 ± | 34.434 ± |
| | | 0.000 | 0.000 | 0.000 | 0.000 | 0.000 |
| | B | 21.656 ± | 21.612 ± | 21.559 ± | 21.513 ± | 21.468 ± |
| | | 0.000 | 0.000 | 0.000 | 0.000 | 0.000 |
| | C | 14.515 ± | 14.457 ± | 14.389 ± | 14.327 ± | 14.267 ± |
| | | 0.000 | 0.000 | 0.000 | 0.000 | 0.000 |
| Empty | A | 31.814 ± | 31.810 ± | 31.800 ± | 31.796 ± | 31.797 ± |
| tubing | | 0.000 | 0.000 | 0.001 | 0.000 | 0.000 |
| | B | 18.937 ± | 18.939 ± | 18.934 ± | 18.933 ± | 18.935 ± |
| | | 0.000 | 0.000 | 0.000 | 0.000 | 0.000 |
| | C | 12.260 ± | 12.262 ± | 12.258 ± | 12.258 ± | 12.259 ± |
| | | 0.000 | 0.000 | 0.000 | 0.000 | 0.000 |

TABLE 4
Weights of tubing sets for formulations
mAb A 50 mg/mL DS and mAb A 150 mg/mL DS

| | | | | Weight at |
|---|---|---|---|---|
| | Tubing | Sampling | Initial weight (g) | sampling time |
| Tubing set | type | time (h) | (average + SD) | (g) (average + SD) |
| mAb A, | A | 24 | 35.166 ± 0.001 | 34.994 ± 0.000 |
| 50 mg/mL | A | 48 | 35.449 ± 0.000 | 35.083 ± 0.000 |
| | A | 72 | 35.076 ± 0.000 | 34.526 ± 0.000 |
| | A | 96 | 35.073 ± 0.000 | 34.355 ± 0.000 |
| | B | 24 | 21.528 ± 0.000 | 21.488 ± 0.000 |
| | B | 48 | 21.528 ± 0.000 | 21.429 ± 0.000 |
| | B | 72 | 21.649 ± 0.000 | 21.513 ± 0.000 |
| | B | 96 | 21.696 ± 0.000 | 21.519 ± 0.000 |
| | C | 24 | 14.771 ± 0.000 | 14.712 ± 0.000 |
| | C | 48 | 14.488 ± 0.000 | 14.366 ± 0.000 |
| | C | 72 | 14.593 ± 0.000 | 14.408 ± 0.000 |
| | C | 96 | 14.555 ± 0.000 | 14.308 ± 0.000 |
| mAb A, | A | 24 | 36.093 ± 0.000 | 35.914 ± 0.000 |
| 150 mg/mL | A | 48 | 35.912 ± 0.000 | 35.534 ± 0.000 |
| | A | 72 | 35.630 ± 0.000 | 35.085 ± 0.000 |
| | A | 96 | 36.275 ± 0.000 | 35.570 ± 0.000 |
| | B | 24 | 22.176 ± 0.000 | 22.136 ± 0.000 |
| | B | 48 | 22.104 ± 0.000 | 22.015 ± 0.000 |
| | B | 72 | 21.975 ± 0.000 | 21.841 ± 0.000 |
| | B | 96 | 21.874 ± 0.000 | 21.699 ± 0.000 |
| | C | 24 | 14.707 ± 0.000 | 14.650 ± 0.000 |
| | C | 48 | 14.707 ± 0.000 | 14.690 ± 0.000 |
| | C | 72 | 14.720 ± 0.000 | 14.536 ± 0.000 |
| | C | 96 | 14.662 ± 0.000 | 14.412 ± 0.000 |

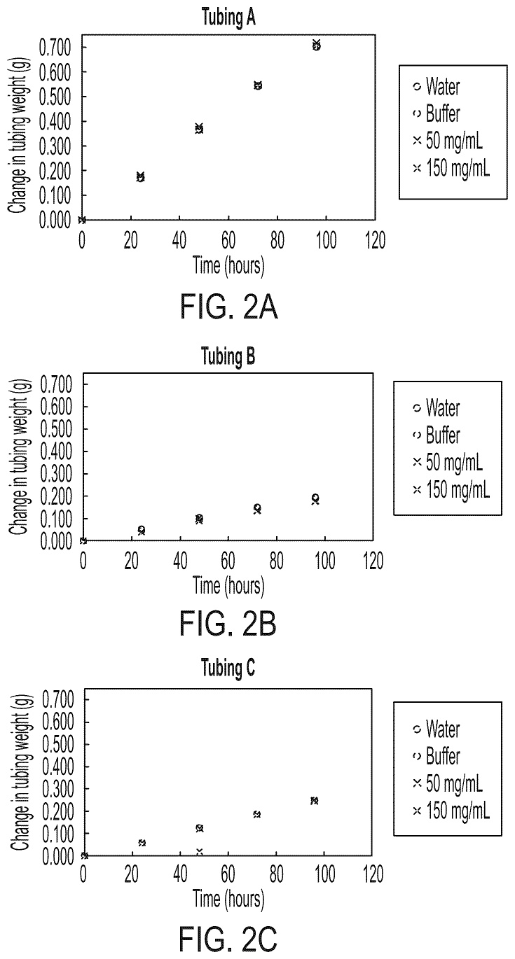

The difference between the initial weight and the weight over time up to 96 hours for the four tubing sets containing water, buffer, mAb A 50 mg/mL DS, and mAb A 150 mg/mL DS was plotted against time for each tubing type, A, B, and C, as shown in FIGS. 2A, 2B and 2C, respectively. Weights were recorded at 24 hours, 48 hours, 72 hours and 96 hours after study set-up. Each tubing set consisted of the three tubing types. One tubing piece of each type was used for the water and buffer control sets, while four tubing pieces of each type corresponding to the four time points were used for the formulation sets. Three weights were recorded per tubing and the average weight was plotted, with the standard deviation being used to plot error bars. The tubings were maintained under ambient temperature (about 22° C.) throughout the study. Relative humidity data was internally recorded by the Facilities Management department at Regeneron. The air circulation in the biosafety cabinet was turned off.

It was observed that the points representing change in tubing weight for all four plotted sets overlap with each other for tubings A, B, and C, suggesting that the rate of weight change is comparable for the four types of solutions for a given tubing type. Linear regression was performed on the data plotted in FIGS. 2A, 2B, and 2C obtained from all four sets for each tubing type, to estimate the rate of change in tubing weight. The slopes, intercepts, and coefficients of determination (R²) obtained for the three tubing types are reported in Table 5. All four solution sets were considered in the regression for each tubing type. It was observed that a linear fit for all tubing types is acceptable as evidenced by the high R² value of >0.9 for each fitting. Overall, it was concluded that the rate of change in tubing weight is dependent only on the tubing type and is independent of the starting concentration of the mAb. Further, the rates of change in weight obtained for the water and buffer controls are comparable to those obtained for the mAb formulations. This observation confirms that the loss in tubing weight over time is attributed to the loss of water from the tubing, as water is the only common component within the contents of all four tubing sets.

TABLE 5
Slope, intercept, and coefficient of determination
(R²) obtained from linear regression of change in tubing
weights (g) as a function of time (h) obtained for the
water, buffer, mAb A 50 mg/mL DS, and mAb A 150 mg/mL
DS-filled tubing sets for each tubing type A, B, and C

| Tubing | Slope | | |
|---|---|---|---|
| type | (g/h) | Intercept | R² |
| A | 0.00745 | 0.00191 | 0.9987 |
| B | 0.00194 | 0.00000 | 0.9915 |
| C | 0.00261 | −0.00167 | 0.9357 |

It was also observed that for the four tubing sets the average rate of change in weight for tubings of types A, B, and C, was 0.0075 g/h, 0.0019 g/h, and 0.0026 g/h, respectively. This observation suggests that the rate of water loss is highest for tubing A, which has a greater SA/V compared to tubings B and C, and higher for tubing C, which has comparable SA/V but smaller wall thickness compared to tubing B. Overall, these results are consistent with the hypothesis that the rate of water loss is dependent on the SA/V and wall thickness of the tubings.

### Example 2. Change in Protein Concentration Over Time for mAb a DS

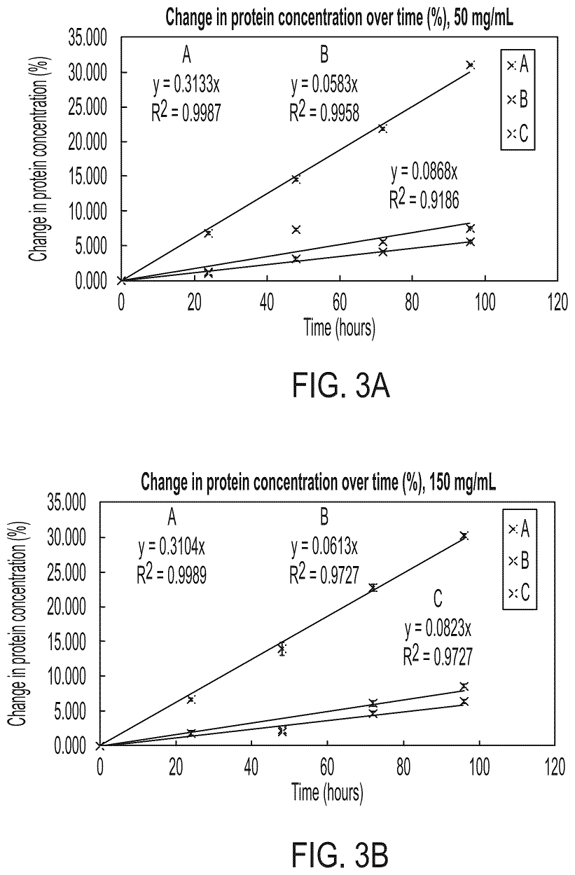

The rate of water loss from silicone tubing was estimated directly from the rate of change in tubing weights, as discussed in the previous example. The rate of water loss can also be indirectly estimated by analyzing mAb A protein concentration as a function of time for the mAb A DS-filled tubing sets. The change in protein concentration (%) obtained via RP-UPLC as a function of time for tubings A, B, and C containing mAb A 50 mg/mL DS and mAb A 150 mg/mL DS-filled tubing sets are plotted in FIG. 3. Each tubing set consisted of three tubing types, A, B, and C. RP-UPLC analysis was performed at 0 hours, 24 hours, 48 hours, 72 hours, and 96 hours after study set-up. Three injections were performed per tubing. The average and standard deviation for each reading was plotted. The tubings were maintained under ambient temperature (about 22° C.) throughout the study. The air circulation in the biosafety cabinet was turned off.

It was observed that the protein concentration increases with time for all tubings. The rate of increase in protein concentration, given by the slope of plots in FIG. 3, was highest for tubing A, followed by tubing C, and least for tubing B, for both mAb A formulations. This trend supports the hypothesis that tubing A with the highest SA/V would undergo a higher rate of water loss compared to tubings B and C, which would further correspond to a higher rate of increase in protein concentration. Interestingly, the rate of increase in protein concentration was comparable for both mAb A formulations for a given tubing type as seen by the comparable slopes, suggesting that both the change in tubing weight and protein concentration (%) is independent of formulation composition.

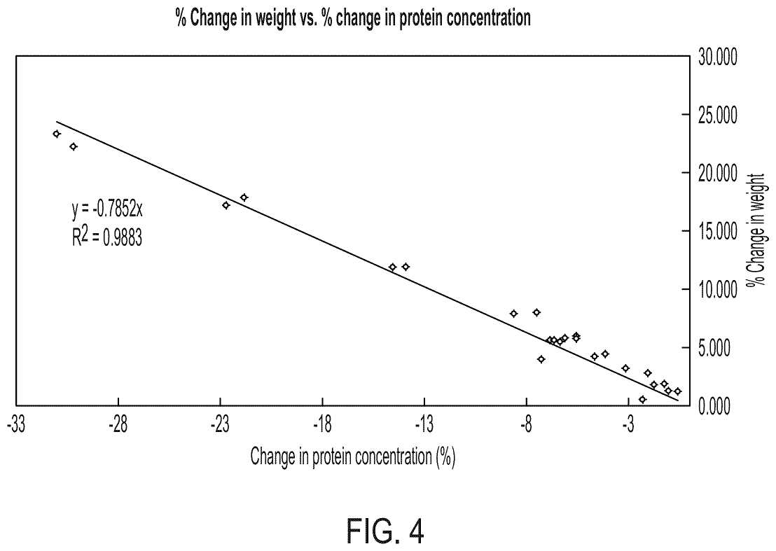

The percentage change in weight of tubing was plotted against percentage change in protein concentration for all tubing types and both mAb A formulation sets of starting concentration 50 mg/mL and 150 mg/mL, as shown in FIG. 4. The percentage change in protein concentration was calculated as the percentage difference in the initial protein concentration and concentration at a given time divided by the initial concentration for a given formulation. The percentage change in weight corresponds to an initial solution volume of 3 mL filled in each tubing. The protein concentration was analyzed using RP-UPLC. Three injections were performed per tubing piece and the average was plotted. Three weight measurements were also taken per tubing piece at a given time point.

The percentage change in tubing weight seemed to vary linearly with the percentage change in protein concentration. A linear regression was applied to the combined data using JMP®, and the significance of the slope was analyzed. The results are shown in Table 6.

TABLE 6
Results from linear regression of the change in tubing
weight (%) plotted against the change in protein
concentration (%) for all tubing types A, B, and
C for both mAb A 50 mg/mL and mAb A 150 mg/mL DS

| | |
|---|---|
| Linear fit | Change in weight (%) = −0.7852 * |
| | Change in protein concentration (%) |
| Total observations | 24 |
| Level of | 0.05 |
| significance | |

Analysis of variance (tested against
model: Change in weight = 0)

| | Degree of | Sum of | Mean | |
|---|---|---|---|---|
| Source | Freedom | squares | square | F Ratio |
| Model | 1 | 2313.1223 | 2313.12 | 1939.407 |
| Error | 23 | 27.4320 | 1.19 | Prob > F |
| Total | 24 | 2340.5543 | | <0.0001 |

Parameter estimates

| Term | Estimate | Std error | t-ratio | Prob > \| t \| |
|---|---|---|---|---|
| Slope | −0.785157 | 0.017829 | −44.04 | <0.0001 |

It was observed that the p-value obtained from the analysis of variance was smaller than the significance level (0.05), suggesting that the linear model is significant. Further, the slope obtained from the fit also has a p-value lower than the significance level, suggesting that the slope is significant.

These results suggest that the relative change in protein concentration per unit mass of water lost is comparable for both mAb formulations and all tubing types. It was concluded from the linear fit obtained for all data that the same mechanism in all tubings results in water loss over time, confirming that water loss is the primary driver for change in protein concentration. Additionally, these results indicate that change in tubing weight is indicative of and can be used as a tracker for the change in protein concentration. Hence, subsequent analysis was performed on tubing weight change.

### Example 3. Effect of Protein Type on Water Loss from Tubing

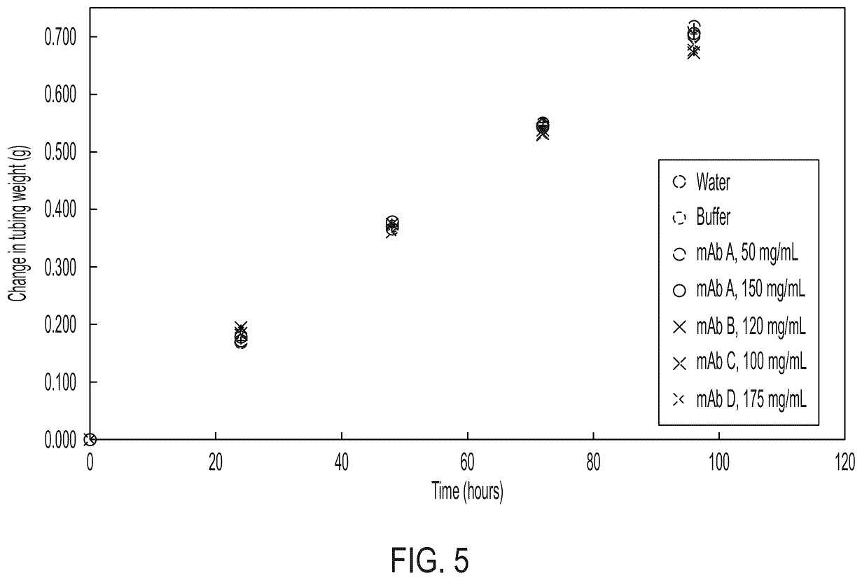

As described in the previous examples, the rate of weight loss in the tubings was found to be independent of the concentration of mAb A and is attributed to water loss from the tubings. To corroborate this conclusion, it was further hypothesized that the rate of water loss is also independent of the type of mAb filled in the tubings. The following formulations with varying protein concentrations and formulation compositions were filled separately in tubing A pieces: mAb B DS, 120 mg/mL; mAb C DS, 100 mg/mL; mAb D DS, 175 mg/mL. Tubing weight change over time was measured for mAb B DS, mAb C DS and mAb D DS, where the weight of the tubing was monitored for up to 96 hours after set-up. The change in weight of the tubing for the various formulations as a function of time is plotted in FIG. 5 along with the change in weights obtained previously for mAb A formulations with concentrations 50 mg/mL and 150 mg/mL, water, and buffer in tubing A. Weights were recorded at 24 hours, 48 hours, 72 hours, and 96 hours after study set-up. All data is shown for formulations filled in tubing A. Three weights were recorded per tubing and the average weight was plotted with the standard deviation being used to plot error bars. The tubings were maintained under ambient temperature (about 22° C.) throughout the study. Relative humidity data was internally recorded by the Facilities Management department in Regeneron. The air circulation in the biosafety cabinet was turned off. It should be noted that the average relative humidity over 96 hours was comparable between the execution period of the previously-conducted study with water, buffer, and mAb A DS (48%) and the study involving other mAbs (52%), allowing comparison between the datasets.

It was observed that the points corresponding to weight change for all formulations overlap with each other, suggesting that the change in weight of the tubings is independent of protein type and concentration. Additionally, it is worth noting that the same change in weight obtained for low-to-moderate protein concentration DSs (mAbs A-C) also takes place in the high-concentration mAb D DS. Overall, it can be concluded that the rate of water loss from the tubings is independent of the protein concentration, protein type, and formulation type. Therefore, the method of the present invention is not limited to a specific protein or protein concentration, but may be applied for a sample including any protein or proteins of interest at any concentration.

### Example 4. Effect of Water Activity on Water Loss from Tubing

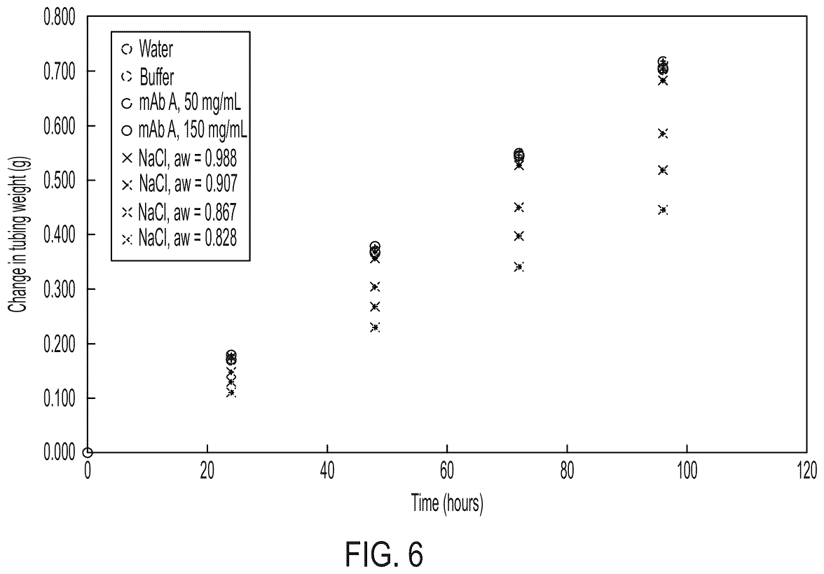

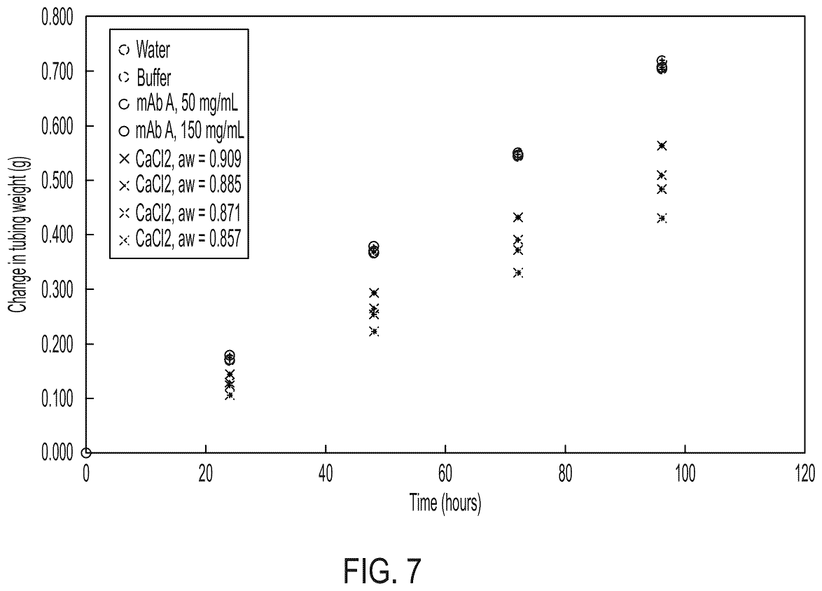

The water activity of a formulation determines the available “free” water concentration in the tubing and can hence potentially influence the rate of water loss. The effect of water activity of the formulation on the rate of water loss was investigated for salt solutions of NaCl and CaCl₂ prepared at specified concentrations. Solutions of NaCl and CaCl₂ of various concentrations by weight were prepared in MilliQ water over a range of initial water activities, and 3 mL of each was filled in separate tubing A pieces. The initial water activity for all solutions was calculated by first calculating the freezing point depression for the solutions from the theoretically calculated osmolality of the solutions and feeding the freezing point depression into the Hildebrand-Scott equation (Equation 15) (Miyawaki et al.). Table 7 shows the concentrations (weight basis) of NaCl and CaCl₂ solutions, calculated initial osmolality, and corresponding initial calculated water activity from the Hildebrand-Scott equation prepared to study the effect of water activity on water loss over time from tubings filled with these salt solutions. The tubings were placed in a biosafety cabinet under ambient temperature (about 22° C.) throughout the study. Relative humidity data was internally recorded by the Facilities Management department in Regeneron. The airflow in the biosafety cabinet was turned off. The change in weight of the tubings over 96 hours was measured at predetermined time points. Three weights were recorded per tubing and the average weight is plotted with the standard deviation being used to plot error bars. The change in tubing weight as a function of time for solutions with varying water activities is shown for NaCl solutions in FIG. 6 and CaCl₂ solutions in FIG. 7, plotted alongside weight change data for pure water, buffer, and mAb A formulations (50 mg/mL and 150 mg/mL) held in tubing A obtained from the previous study. It should be noted that the average relative humidity over 96 hours was comparable between the execution period of the study conducted in Example 1(48%) and salt solution study in Example 4 (44%), allowing comparison between the datasets.

It was found that the rate of change in tubing weight with tubing water activity aw_{tubing}<0.988 collectively for all NaCl and CaCl₂ solutions varies from that observed for the mAb A formulations (aw_{tubing} for mAb A, 150 mg/mL DS=0.991, aw_{tubing} for mAb A, 50 mg/mL DS=0.992), water (aw_{tubing}=1), and buffer (aw_{tubing}=0.993). The rate of water loss increased with an increase in the water activity for both NaCl and CaCl₂ solutions.

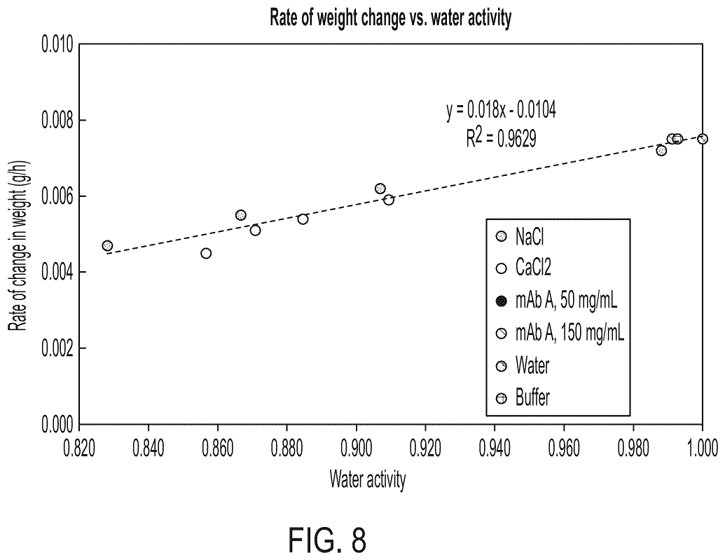

Next, the rates of change of weight obtained from the slopes in FIG. 6 and FIG. 7 for NaCl and CaCl₂ salt solutions and in FIG. 2 for mAb A protein solutions, buffer, and water held in tubing A were plotted as a function of the initial water activity calculated through the Hildebrand-Scott equation, as shown in FIG. 8. It was found that a linear relationship with an R2 of about 0.96 exists between the rate of weight loss and initial water activity for all solutions. Hence, it was concluded that the rate of water loss is a linear function of initial water activity, regardless of the nature of the solute.

Further, it was observed from FIG. 6 that the rate of weight change for NaCl-filled tubing has a weak dependence on the water activity above initial activity values greater than 0.988. From Table 7, the corresponding osmolality of the 1.91% (w/w) NaCl solution with a water activity of 0.988 is 667 mOsm/kg. This value is comparably higher than the typical average osmolality of protein formulations used for parenteral administration to maintain isotonic conditions (Banks et al., 2018, ProteinScience, 27(12):2073-2083). Hence, it can be concluded that the effect of water activity on the rate of water loss in the high-activity protein formulations used in this study is expected to be negligible. Nevertheless, the water activity is considered an important parameter in determining the rate of water loss in subsequent modeling calculations (Equations 1-16).

TABLE 7
Salt solution characteristics

| | NaCl | Water | | CaCl₂ | Water |
|---|---|---|---|---|---|
| Concentration | solution | activity | Concentration | solution | activity |
| (% w/w) of | osmolality | of NaCl | (% w/w) of | osmolality | of CaCl₂ |
| NaCl | (mOsm/kg) | solution | CaCl₂ | (mOsm/kg) | solution |
| 1.91 | 668 | 0.988 | 16.27 | 5253 | 0.909 |
| 13.64 | 5406 | 0.907 | 20.04 | 6775 | 0.885 |
| 18.76 | 7900 | 0.867 | 22.02 | 7635 | 0.871 |
| 23.30 | 10394 | 0.828 | 24.01 | 8540 | 0.857 |

### Example 5. Mechanistic Modeling of Water Loss Through Tubing

Mass transfer of water occurring from the silicone tubings is expected to be mainly diffusive in nature because of the absence of bulk fluid flow, pressure, or other sources of convective transfer. Fick's first law (Bird et al.) was hypothesized to describe this mass transfer phenomenon and was used to develop a mechanistic model that can estimate the weight loss occurring in formulation-filled tubings. The development of a mechanistic model is useful to explain data obtained from this study, as well as being beneficial for future applications, for example small scale process development studies and large-scale filling operations.

The following steps were taken for mechanistic modeling of Fick's 1^{st} law: (1) First, experimental data was used to generate the effective diffusivity (D) for the studied tubings. D through Fick's 1^{st} law (Equation 11) and the water activity using the Hildebrand-Scott equation (Equation 15) were calculated for each time interval and formulation-tubing combination investigated in the studies in Examples 1 and 4. The following tubing-formulation combinations were included in the study: tubings A, B, C containing water, buffer, and mAb A formulations each; and tubing A containing NaCl and CaCl₂ solutions at various concentrations; (2) an empirical relationship between D and water activity (aw_{tubing}), referred to as Equation 17, was established from the combined data from these formulation-tubing combinations, and variability in D was accounted for in Equation 17 through applying 99% prediction intervals; (3) Next, Equation 20 was used to predict the rate of water loss by using the obtained empirical relationship for D and aw_{tubing}. Finally, Equation 18 was used to calculate the weight change over a given time period, Δt (Equations 18-21, Equations 1-16); and (4) Model verification was performed using tubings with ID and wall thickness differing from those used to develop the relationship between D and awtᵤbi_{ng}. Tubing weights were experimentally recorded over time and also calculated using the derived model. The upper and lower limits of the range of calculated water loss obtained were compared to the experimentally obtained water loss for the specified mAb-tubing combinations.

The effective diffusivity constant (D) calculated from experimental data at each time points described in Step 1 is reported in Table 8 along with the corresponding water activity for: (1) each tubing A, B, and C containing water, buffer, mAb A 50 mg/mL DS, and mAb A 150 mg/mL DS; and (2) each tubing A containing NaCl and CaCl₂ solutions.

The average D for mAb A, buffer, and water sets is reported for each tubing type in Table 9 along with the standard deviation. The overall average D across all four formulations and three tubing types is also reported in Table 9. The average effective diffusivity was calculated from effective diffusivity values obtained for four formulation types: water, buffer, mAb A 50 mg/mL DS, and mAb A 150 mg/mL DS.

TABLE 8
Effective diffusivity of water calculated using Fick's first
law for: (1) each tubing A, B and C containing water, buffer,
mAb A 50 mg/mL DS, and mAb A 150 mg/mL DS; (2) each tubing
A containing NaCl and CaCl2 solutions of various concentrations.
The effective diffusivity calculated at different time points
is presented. The corresponding water activity at that time
calculated using Equations 12-15 is also reported.

| | | | | Time | | Effective |
|---|---|---|---|---|---|---|
| Tubing | Solution | Concentration | | (h) | aw_{tubing} | D (m²/s) |
| A | mAb A | 50 | mg/mL | 24 | 0.992 | 8.81E−13 |
| | | | | 48 | 0.991 | 9.73E−13 |
| | | | | 72 | 0.990 | 9.45E−13 |
| | | | | 96 | 0.990 | 9.86E−13 |
| B | mAb A | 50 | mg/mL | 24 | 0.992 | 9.13E−13 |
| | | | | 48 | 0.992 | 1.28E−12 |
| | | | | 72 | 0.992 | 7.92E−13 |
| | | | | 96 | 0.992 | 9.15E−13 |
| C | mAb A | 50 | mg/mL | 24 | 0.992 | 8.92E−13 |
| | | | | 48 | 0.992 | 9.29E−13 |
| | | | | 72 | 0.992 | 8.80E−13 |
| | | | | 96 | 0.991 | 9.47E−13 |
| A | mAb A | 150 | mg/mL | 24 | 0.991 | 9.16E−13 |
| | | | | 48 | 0.990 | 1.01E−12 |
| | | | | 72 | 0.989 | 8.65E−13 |
| | | | | 96 | 0.988 | 9.39E−13 |
| B | mAb A | 150 | mg/mL | 24 | 0.991 | 9.05E−13 |
| | | | | 48 | 0.991 | 1.07E−12 |
| | | | | 72 | 0.991 | 9.51E−13 |
| | | | | 96 | 0.991 | 9.18E−13 |
| C | mAb A | 150 | mg/mL | 24 | 0.991 | 8.81E−13 |
| | | | | 48 | 0.991 | 9.04E−13 |
| | | | | 72 | 0.991 | 8.62E−13 |
| | | | | 96 | 0.990 | 1.00E−12 |
| A | Buffer | — | | 24 | 0.992 | 8.81E−13 |
| | | | | 48 | 0.992 | 9.93E−13 |
| | | | | 72 | 0.991 | 9.08E−13 |
| | | | | 96 | 0.991 | 9.30E−13 |
| B | Buffer | — | | 24 | 0.993 | 1.02E−12 |
| | | | | 48 | 0.993 | 1.14E−12 |
| | | | | 72 | 0.992 | 9.62E−13 |
| | | | | 96 | 0.992 | 1.03E−12 |
| C | Buffer | — | | 24 | 0.993 | 8.95E−13 |
| | | | | 48 | 0.992 | 9.76E−13 |
| | | | | 72 | 0.992 | 8.79E−13 |
| | | | | 96 | 0.992 | 9.15E−13 |
| A | Water | — | | 24 | 1.000 | 8.58E−13 |
| | | | | 48 | 1.000 | 9.72E−13 |
| | | | | 72 | 1.000 | 8.91E−13 |
| | | | | 96 | 1.000 | 9.11E−13 |
| B | Water | — | | 24 | 1.000 | 1.16E−12 |
| | | | | 48 | 1.000 | 1.09E−12 |
| | | | | 72 | 1.000 | 9.87E−13 |
| | | | | 96 | 1.000 | 9.92E−13 |
| C | Water | — | | 24 | 1.000 | 8.81E−13 |
| | | | | 48 | 1.000 | 9.52E−13 |
| | | | | 72 | 1.000 | 8.82E−13 |
| | | | | 96 | 1.000 | 9.14E−13 |
| A | Aq. NaCl | 1.92 | wt % | 24 | 0.987 | 8.77E−13 |
| | | | | 48 | 0.986 | 8.92E−13 |
| | | | | 72 | 0.986 | 8.23E−13 |
| | | | | 96 | 0.985 | 7.90E−13 |
| A | Aq. NaCl | 13.64 | wt % | 24 | 0.902 | 8.86E−13 |
| | | | | 48 | 0.896 | 9.07E−13 |
| | | | | 72 | 0.890 | 8.15E−13 |
| | | | | 96 | 0.884 | 7.96E−13 |
| A | Aq. NaCl | 18.76 | wt % | 24 | 0.861 | 8.62E−13 |
| | | | | 48 | 0.854 | 8.77E−13 |
| | | | | 72 | 0.846 | 7.84E−13 |
| | | | | 96 | 0.839 | 7.63E−13 |
| A | Aq. NaCl | 23.30 | wt % | 24 | 0.822 | 8.17E−13 |
| | | | | 48 | 0.814 | 8.34E−13 |
| | | | | 72 | 0.806 | 7.25E−13 |
| | | | | 96 | 0.798 | 7.16E−13 |
| A | Aq. CaCl₂ | 16.27 | wt % | 24 | 0.905 | 8.60E−13 |
| | | | | 48 | 0.900 | 8.57E−13 |
| | | | | 72 | 0.894 | 7.66E−13 |
| | | | | 96 | 0.888 | 7.54E−13 |
| A | Aq. CaCl₂ | 20.04 | wt % | 24 | 0.879 | 8.21E−13 |
| | | | | 48 | 0.873 | 8.22E−13 |
| | | | | 72 | 0.867 | 7.25E−13 |
| | | | | 96 | 0.861 | 7.10E−13 |
| A | Aq. CaCl₂ | 22.02 | wt % | 24 | 0.866 | 8.13E−13 |
| | | | | 48 | 0.859 | 8.16E−13 |
| | | | | 72 | 0.853 | 6.99E−13 |
| | | | | 96 | 0.847 | 6.83E−13 |
| A | Aq. CaCl₂ | 24.00 | wt % | 24 | 0.851 | 7.25E−13 |
| | | | | 48 | 0.845 | 7.53E−13 |
| | | | | 72 | 0.839 | 6.50E−13 |
| | | | | 96 | 0.833 | 6.23E−13 |

TABLE 9
Average effective diffusivity of water calculated using
Fick's first law for each tubing type A, B, and C

Average effective Diffusivity
(×10⁻¹³ m²/s) (all formulations combined)

| Tubing | | | | Combined A, |
|---|---|---|---|---|
| type | A | B | C | B, and C |
| Average | 9.3 | 10.1 | 9.1 | 9.5 |
| % Standard | 1.7 | 4.7 | 0.4 | 5.4 |
| deviation | | | | |

It was observed that the average effective diffusivity for all tubings was 9.5*10⁻¹³ m²/s, comparable to values in literature for diffusivity of water through a porous membrane or material that are of the order of 10⁻¹¹-10⁻¹³ m²/s (Fasano et al., 2016, Nat Commun, 7:12762; Hoch et al., 2003, Journal of Membrane Science, 214(2):199-209).

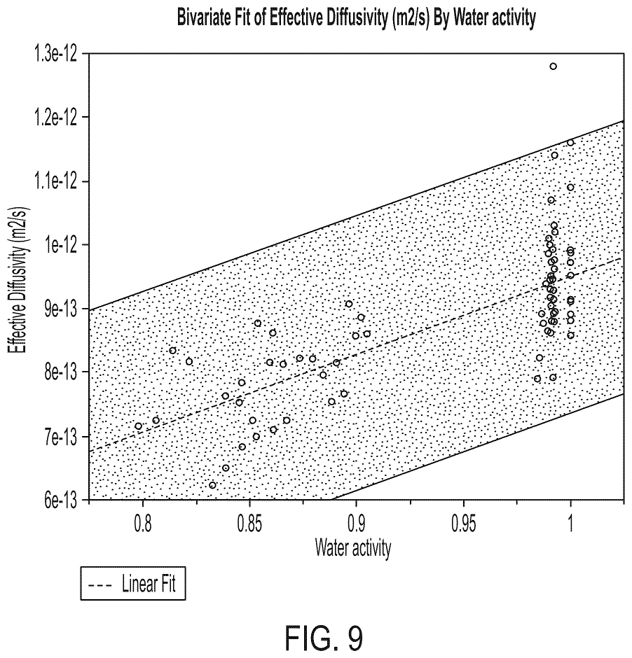

Next, the effective diffusivity values obtained for the protein and salt solutions were plotted against the water activity for all time points for each tubing-formulation combination. A linear regression was performed between D and aw_{tubing}, as shown in FIG. 9. Further, the 99% prediction interval for an individual predicted value of D was also plotted using JMP® in FIG. 9, indicated by the shaded area. The fit parameters associated with the linear regression between D and aw_{tubing} are shown in Table 10. The formulation-tubing combinations include: mAb A (50 mg/mL and 15 mg/mL) DS, water, and buffer held in tubings A, B, C; and salt solutions (NaCl and CaCl₂)) of various concentrations held in tubing A. The effective diffusivity and water activity values were calculated for each formulation-tubing combination at various time points. Linear regression was performed using JMP®. It was found that the p-value associated with the analysis of variance was <0.0001, compared to the significance level of 0.05, suggesting that the linear fit was significant. This was corroborated by the p-value of <0.0001 associated with the slope of the fit. The following relationship was established between D and aw_{tubing}:

$$D ( \frac{{m}^{2}}{s} ) = - 2.69 * 1 {0}^{- 13} + 1.22 * 1 {0}^{- 12} * {aw}_{tubing} \qquad ( 17 )$$

The upper and lower 99% prediction limits that bind the shaded region shown in FIG. 9 were also established. The linear relationships obtained are shown in Table 11. The equations follow from the linear regression performed between D and aw_{tubing} in FIG. 9. The following data points were used in generating the plot: mAb A (50 mg/mL and 15 mg/mL) DS, water, and buffer held in tubings A, B, C; and salt solutions of various concentrations (NaCl and CaCl2) held in tubing A.

TABLE 10
Results from the linear regression of the effective diffusivity
of water in various formulation-tubing combinations plotted
against their corresponding water activity

| | |
|---|---|
| Linear fit | Effective diffusivity (m²/s) = −2.69 * |
| | 10⁻¹³ + 1.22 * 10⁻¹² * Water activity |

| | | |
|---|---|---|
| Summary | R² | 0.507298 |
| of fit | | |
| | R² adjusted | 0.500981 |
| | Room Mean Square Error | 8.02e−14 |
| | Mean of Response | 8.85e−13 |

| | |
|---|---|
| Total | 80 |

observations

| | |
|---|---|
| Level of | 0.05 |

significance

Analysis of variance (tested against model:
Effective diffusivity = 0)

| | Degree of | Sum of | Mean | |
|---|---|---|---|---|
| Source | Freedom | squares | square | F Ratio |
| Model | 1 | 5.1648e−25 | 5.165e−25 | 80.3107 |
| Error | 78 | 5.0162e−25 | 6.431e−27 | Prob > F |
| Total | 79 | 1.0181e−24 | | <0.0001 |

Parameter estimates

| Term | Estimate | Std error | t-ratio | Prob > \| t \| |
|---|---|---|---|---|
| Intercept | −2.69e−13 | 1.29e−13 | −2.08 | 0.0405 |
| Slope | 1.22e−12 | 1.36e−13 | 8.96 | <0.0001 |

TABLE 11
Linear relationships obtained for the upper and lower
99% prediction intervals for an individual predicted
value of D, established between D and aw_{tubing}

| | Linear relationship |
|---|---|
| Lower 99% prediction interval | D (m²/s) = 1.236*10⁻¹² * |
| | aw_{tubing} − 4.99*10⁻¹³ |
| Upper 99% prediction interval | D (m²/s) = 1.203*10⁻¹² * |
| | aw_{tubing} − 3.90*10⁻¹⁴ |

Next, these relationships between D and aw_{tubing} were used to calculate the upper and lower bounds of the expected weight loss from tubing as a function of time for given tubing dimensions and formulation properties. An iterative process was employed for calculating the weight loss after a specified time interval. The assumptions stated in the Data Analysis section were used. Equation 18 shows the model used:

$$Δ Weight ( t ) ( g ) = - {B}^{′} ( t ) ( \frac{g}{s} ) * Δ t ( s ) \qquad ( 18 )$$

Where,
Δt (s)=Time period for weight loss calculation; ΔWeight (t) (g)=Change in weight calculated over time period zit from t to t+Δt h;

$${B}^{′} ( t ) ( \frac{g}{s} ) = Rate of weight loss over time period Δ t ;$$

Substituting Equations 3 and 5 for ΔWeight (t) in Equation 18,

$${J}_{{water}_{Δ t}} ( \frac{g}{{m}^{2} s} ) * Δ t ( s ) * \frac{4 V ( t )}{ID} ( {m}^{2} ) = - {B}^{′} ( t ) ( \frac{g}{s} ) * Δ t ( s ) \qquad ( 19 )$$

### Where,

J_{water}_{_{Δt}}=Incremental flux of water between t and (t+Δt) h;
V(t)=Solution volume in tubing at time t (m³);
ID=Tubing internal diameter (m);

Substituting Equation 11 for the flux and Equation 17 for D (t) in Equation 19, can be calculated as shown in Equation 20:

$${B}^{′} ( t ) ( \frac{g}{s} ) = \frac{\begin{matrix}D ( t ) ( \frac{{m}^{2}}{s} ) * {C}_{{water}_{pure}} * [ {aw}_{tubing} ( t ) - \\ {aw}_{ambient} ( t ) ] ( \frac{g}{{m}^{3}} ) * \frac{4 V ( t )}{ID} ( {m}^{2} )\end{matrix}}{Δ x ( m )} \qquad ( 20 )$$

Where D(t) is calculated using the relationship between D(t) and aw(t) established in Equation 17 using experimental data.

The approach used in mechanistic modeling of the expected weight loss over interval Δt is calculating B′ at time t using Equation 20 and applying in Equation 18. At study set-up (time=t₀), the known solution osmolality is used to calculate aw_{tubing} using Equations 14 and 15. The relative humidity in the ambient air over time interval zit is measured to provide aw_{ambient} according to Equation 16. The known filled solution volume and tubing ID is used to calculate the surface area exposed to the tubing, 4V/ID using Equation 5. The upper and lower bound relationships between D and aw_{tubing} listed in Table S6 are used to calculate upper and lower values of D as a function of aw_{tubing}. Setting Δt=t₁−t₀, these parameters feed into Equations 18 and 20 to give upper and lower bounds of weight loss from study set-up t₀ to time t₁.

Next, each of the upper and lower bound weight losses over (t₁−t₀) hours is used to calculate an updated solution volume at t₁ hours using Equations 4 and 6. The updated solution volume is used to calculate the updated surface area of solution in contact with tubing using Equation 5. Incorporating Equations 12-15, the aw_{tubing} of the solution at t₁ hours is calculated. The new aw_{tubing} is used to calculate D at t₁ hours using the relationships in Table S6. The aw_{ambient} is calculated according to the method used in the previous paragraph for the period from t₁ to t₂ hours. Hence, the upper and lower bounds of the expected weight loss from t₁ hours to t₂ hours may be calculated. These calculations may be repeated iteratively up to the study end time to develop upper and lower bounds of a weight loss profile over time for a given formulation-tubing combination. Manual measurements may be taken as frequent as every five minutes to provide additional datapoints and the process may potentially be automated to continuously take measurements and make calculations in real-time. The additional data points may be used to validate and develop detailed weight loss profiles.

The change in tubing weight can be converted to a change in protein concentration using the following relationship:

Change in weight (%)=−0.7852*Change in protein concentration (%) (21)

### Example 6. Model Verification

The mechanistic model developed in Example 5 was tested by comparing calculations to experimentally measured weight loss from platinum-cured silicone tubings. The following tubings were used for verification: 0.8 mm ID tubing with 1.6 mm ID wall thickness; 1.2 mm ID tubing with 1.6 mm ID wall thickness; 3.2 mm ID tubing with 1.8 mm ID wall thickness. Three formulations with two types of mAb and covering a range of protein concentrations (mAb A DS (50 mg/mL); mAb A DS (150 mg/mL); and mAb D DS (175 mg/mL)) were filled in each type of tubing. Overall, the following nine formulation-tubing combinations were analyzed: (a) 0.5 mL mAb A (50 mg/mL) DS in 0.8 mm ID tubing; (b) 3 mL mAb A (50 mg/mL) DS in 1.2 mm ID tubing; (c) 3 mL mAb A (50 mg/mL) DS in 3.2 mm ID tubing; (d) 0.5 mL mAb A (150 mg/mL) DS in 0.8 mm ID tubing; (e) 3 mL mAb A (150 mg/mL) DS in 1.2 mm ID tubing; (f) 3 mL mAb A (150 mg/mL) DS in 3.2 mm ID tubing; (g) 0.5 mL mAb D (175 mg/mL) DS in 0.8 mm ID tubing; (h) 3 mL mAb D (175 mg/mL) DS in 1.2 mm ID tubing; (i) 3 mL mAb D (175 mg/mL) DS in 3.2 mm ID tubing. Each formulation was filled in the tubing, sealed, and placed in a biosafety cabinet with the air circulation and lights turned off. The weight of each tubing was measured at study set-up and subsequently every 24 hours. Tubings a-c and g-i were monitored for up to 168 hours while tubings d-f were monitored for up to 96 hours. Weight measurements were conducted in triplicate. In parallel, theoretically expected upper and lower weight loss limits as a function of time based on initial mAb properties and filled volumes were calculated for each formulation-tubing combination using the mechanistic model. The relative humidity measured during the study duration was averaged as shown in Equation 16 over every 24 hours and was used for estimating the theoretical weight loss.

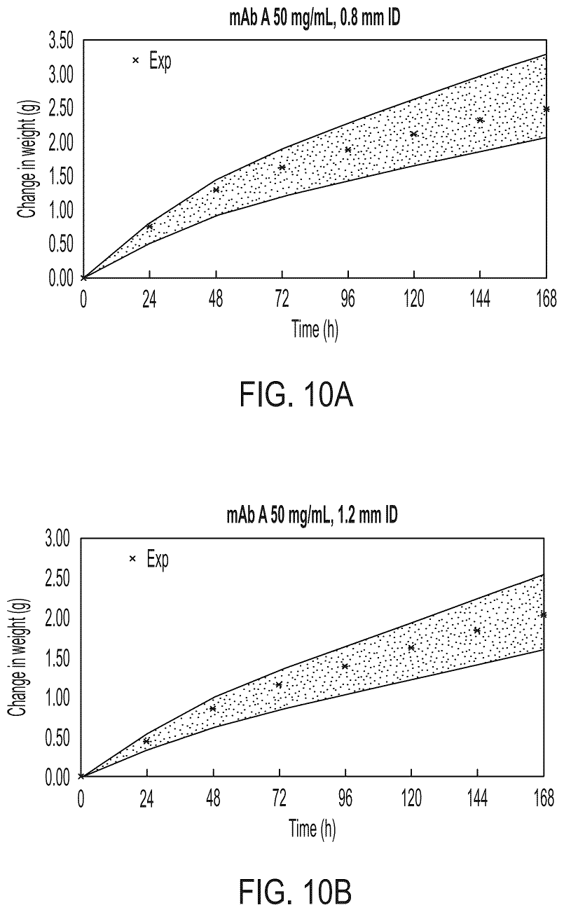

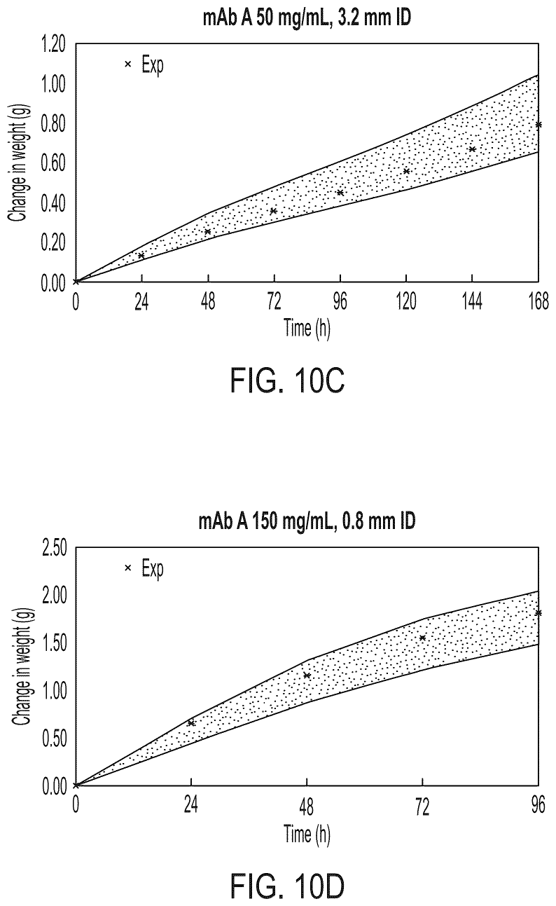

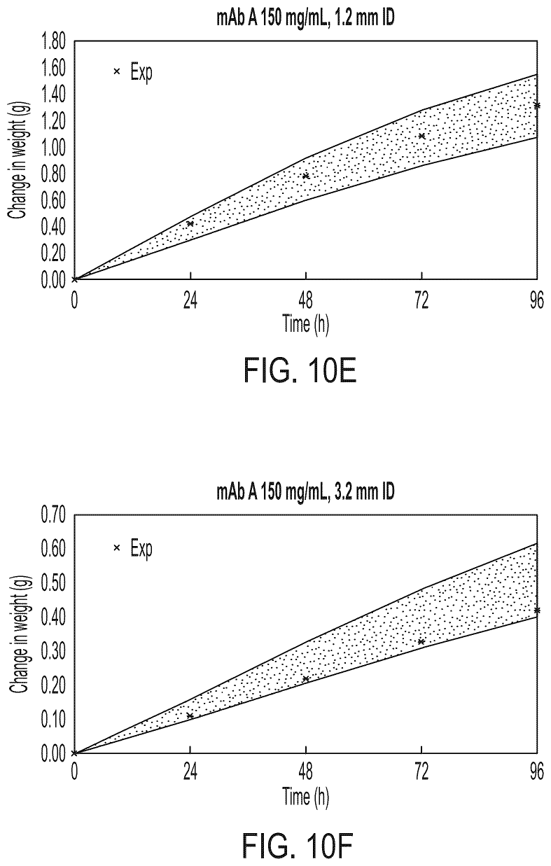

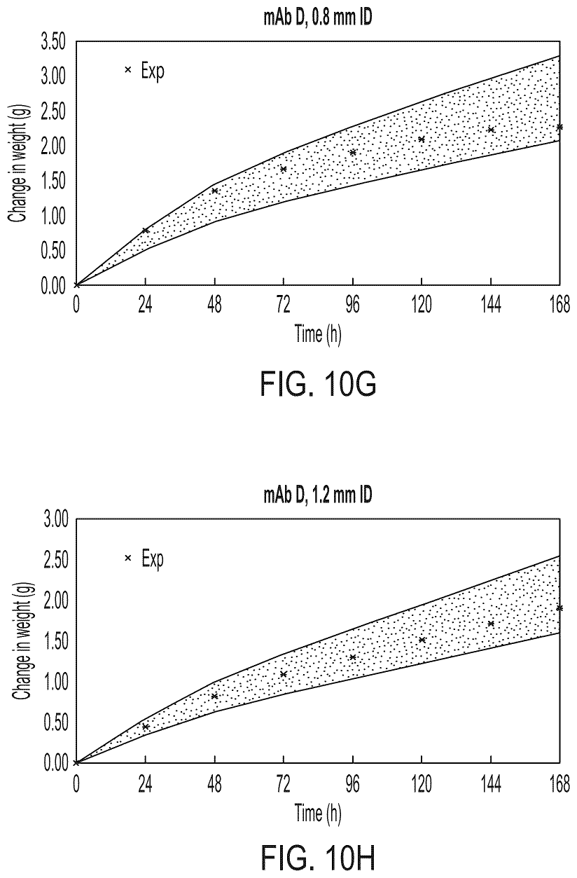

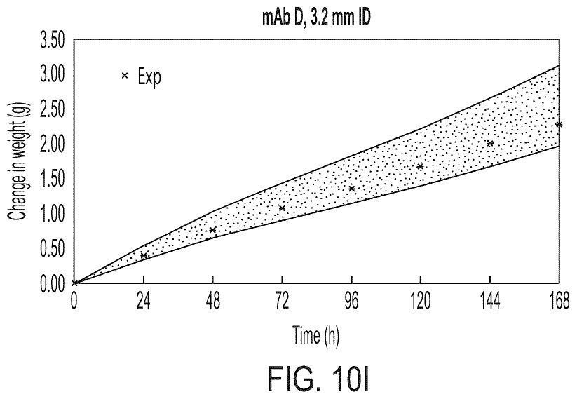

The range of predicted weight loss and the experimental weight loss was plotted against each time point in the study for the nine formulation-tubing combinations, as shown in FIG. 10. Irrespective of actual volume filled at the beginning, all weight losses were normalized to a starting volume of 3 mL. Each plot shows a shaded area that correlates to the range of expected tubing weight loss, with theoretical weight loss limits calculated using 99% prediction levels for water effective diffusivity. Corresponding experimental data is plotted as black scatter crosses. The calculated and experimental weights are also listed in Tables 11A-C. Weights were recorded at 24 hours, 48 hours, 72 hours, and 96 hours after study set-up for FIGS. 10A-10C and 10G-10I, and further every 24 hours up to 168 hours for FIG. 10D-10F. The average of three recorded weights is plotted for each tubing and the standard deviation is plotted as error bars. The tubings were placed in a biosafety cabinet under ambient temperature (about 22° C.) throughout the study. Relative humidity data was internally recorded and used for data processing. The relative humidity measured during the study was averaged every 24 hours to calculate the theoretical weight change. The air circulation inside the biosafety cabinet was turned off.

TABLE 12A
Model-derived theoretical weight loss and experimentally
obtained weight loss at various time points for:
(a) 0.5 mL mAb A (50 mg/mL) DS in 0.8 mm ID tubing;
(b) 3 mL mAb A (50 mg/mL) DS in 1.2 mm ID tubing;
(c) 3 mL mAb A (50 mg/mL) DS in 3.2 mm ID tubing.

| | Weight loss (g) | | | | | | | | |
|---|---|---|---|---|---|---|---|---|---|
| | Sample | | | | | | | | |
| Time | a | | | b | | | c | | |
| (h) | E | L | H | E | L | H | E | L | H |
| 0 | 0 | 0 | 0 | 0 | 0 | 0 | 0 | 0 | 0 |
| 24 | 0.76 | 0.51 | 0.81 | 0.45 | 0.34 | 0.54 | 0.13 | 0.11 | 0.18 |
| 48 | 1.29 | 0.91 | 1.45 | 0.86 | 0.63 | 0.99 | 0.26 | 0.22 | 0.34 |
| 72 | 1.63 | 1.2 | 1.9 | 1.16 | 0.84 | 1.34 | 0.36 | 0.3 | 0.48 |
| 96 | 1.89 | 1.43 | 2.28 | 1.39 | 1.03 | 1.64 | 0.45 | 0.38 | 0.61 |
| 120 | 2.12 | 1.65 | 2.63 | 1.63 | 1.22 | 1.94 | 0.56 | 0.47 | 0.74 |
| 144 | 2.32 | 1.87 | 2.97 | 1.84 | 1.41 | 2.24 | 0.67 | 0.56 | 0.88 |
| 168 | 2.48 | 2.06 | 3.29 | 2.04 | 1.6 | 2.54 | 0.79 | 0.65 | 1.04 |

TABLE 12B
Model-derived theoretical weight loss and experimentally
obtained weight loss at various time points for: (d) 0.5 mL
mAb A (150 mg/mL) DS in 0.8 mm ID tubing; (e) 3
mL mAb A (150 mg/mL) DS in 1.2 mm ID tubing; (f) 3
mL mAb A (150 mg/mL) DS in 3.2 mm ID tubing.

| | Weight loss (g) | | | | | | | | |
|---|---|---|---|---|---|---|---|---|---|
| | Sample | | | | | | | | |
| Time | d | | | e | | | f | | |
| (h) | E | L | H | E | L | H | E | L | H |
| 0 | 0 | 0 | 0 | 0 | 0 | 0 | 0 | 0 | 0 |
| 24 | 0.65 | 0.45 | 0.71 | 0.42 | 0.3 | 0.47 | 0.11 | 0.1 | 0.16 |
| 48 | 1.16 | 0.87 | 1.31 | 0.78 | 0.6 | 0.92 | 0.22 | 0.21 | 0.33 |
| 72 | 1.55 | 1.22 | 1.75 | 1.08 | 0.86 | 1.28 | 0.33 | 0.31 | 0.48 |
| 96 | 1.81 | 1.48 | 2.04 | 1.31 | 1.07 | 1.55 | 0.42 | 0.4 | 0.61 |
| 120 | — | — | — | — | — | — | — | — | — |
| 144 | — | — | — | — | — | — | — | — | — |
| 168 | — | — | — | — | — | — | — | — | — |

TABLE 12C
Model-derived theoretical weight loss and experimentally obtained
weight loss at various time points for: (g) 0.5 mL mAb D (175 mg/
mL) DS in 0.8 mm ID tubing; (h) 3 mL mAb D (175 mg/mL) DS in 1.2
mm ID tubing; (i) 3 mL mAb D (175 mg/mL) DS in 3.2 mm ID tubing.

| | Weight loss (g) | | | | | | | | |
|---|---|---|---|---|---|---|---|---|---|
| | Sample | | | | | | | | |
| Time | g | | | h | | | i | | |
| (h) | E | L | H | E | L | H | E | L | H |
| 0 | 0 | 0 | 0 | 0 | 0 | 0 | 0 | 0 | 0 |
| 24 | 0.79 | 0.51 | 0.81 | 0.44 | 0.34 | 0.54 | 0.4 | 0.34 | 0.54 |
| 48 | 1.35 | 0.91 | 1.45 | 0.82 | 0.63 | 0.99 | 0.77 | 0.65 | 1.03 |
| 72 | 1.67 | 1.2 | 1.9 | 1.09 | 0.84 | 1.34 | 1.07 | 0.91 | 1.44 |
| 96 | 1.9 | 1.44 | 2.28 | 1.3 | 1.03 | 1.64 | 1.36 | 1.15 | 1.82 |
| 120 | 2.09 | 1.66 | 2.63 | 1.51 | 1.22 | 1.94 | 1.68 | 1.4 | 2.22 |
| 144 | 2.23 | 1.87 | 2.97 | 1.71 | 1.41 | 2.24 | 2.01 | 1.67 | 2.66 |
| 168 | 2.27 | 2.07 | 3.29 | 1.91 | 1.6 | 2.54 | 2.28 | 1.97 | 3.12 |

It was found that the experimental weight loss fell within the range of the model-derived weight loss for all the formulation-tubing combinations at each time point, as shown in FIG. 10. This suggests that the experimental and calculated weight loss align across a range of protein concentrations and that the experimental water loss occurring over long hold periods from formulation-filled platinum-cured silicone tubing can be successfully predicted using the derived Fick's 1^{st} law-based mechanistic model. Additionally, it should also be noted that model derivation and verification were performed using platinum-cured silicone tubing from different manufacturers. The alignment between the experimental and predicted weight change suggests that the tubing vendor does not have an effect on the rate of water loss provided the material used is the same. It should be noted that additional factors including the temperature and air flow that were not precisely controlled may also impact the rate of water loss.

### Example 8. Effect of Tubing Construction Material on the Rate of Water Loss

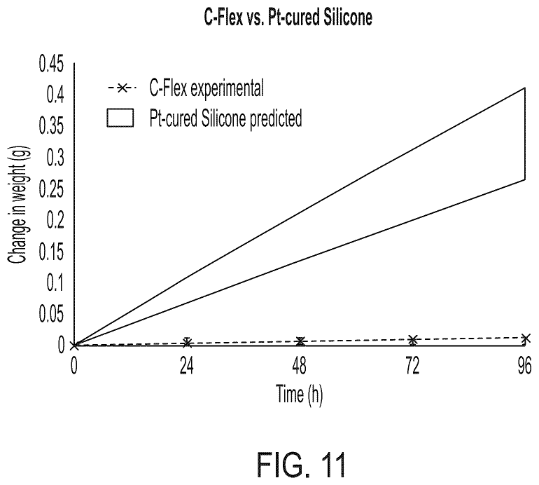

Changing the material of the tubing can potentially impact the rate of diffusion of water, depending on the permeability of the material. In a study that tested the rate of evaporation of water from catheters made of various materials, it was previously found that silicone is highly permeable compared to materials including polyurethane and polyethylene (Instech Blog). Hence, it was hypothesized that platinum-cured silicone tubing would be more permeable to water as compared to C-Flex tubing (polymeric thermoplastic elastomer), for example. This hypothesis was tested by studying the weight loss in C-Flex tubing (3.2 mm ID, 1.6 mm thickness) filled with mAb A DS of protein concentration 150 mg/mL. The tubing was filled in duplicate. Three weights were recorded per tubing. The average observed change in weight for the C-Flex tubing was compared to the range of change in weight predicted through the diffusion-based model for platinum-cured silicone tubing filled with mAb A, 150 mg/mL DS having comparable dimensions (3.2 mm ID, 1.6 mm thickness). A 99% prediction interval was used, and the comparison is shown in FIG. 11. The standard deviation of the weights of two tubings was used to plot error bars for the C-flex tubing. The tubings were placed in a biosafety cabinet under ambient temperature (˜22° C.) throughout the study, with airflow turned off. Relative humidity data was internally recorded by the Facilities Management department in Regeneron.

It was found that the change in weight of the C-Flex tubing was considerably lower than the weight change range predicted for Pt-cured silicone tubing of similar dimensions. Overall, this result suggests that the permeability of Pt-cured silicone to water is higher than that of C-Flex tubing. Therefore, the use of C-Flex material during filling compared to Pt-cured silicone may reduce water loss from tubings during holds. This result demonstrates that the present invention can be used to select a container on the basis of reducing the predicted water loss of a sample. This result further demonstrates that the method of the present invention may be applied to any permeable material used for tubing or for a container.

Silicone tubing is predominantly used in the pharmaceutical industry for several unit operations and hence determining its effect on protein formulations is beneficial for pharmaceutical process development. Silicone tubing is known to be semi-permeable, through which water diffusion can occur. It was hypothesized that the rate of water loss from platinum-cured silicone tubing is a function of the tubing parameters. The weight of platinum-cured silicone tubings filled with several mAb formulations was monitored. The change in protein concentration as a function of time was also analyzed for some tubings. The Examples described above confirm that water loss occurs from protein formulations over hold times through silicone tubings, as evidenced by a loss in weight as well as an increase in protein concentration. Further, the rate of water loss is independent of protein type, concentration, and other typical solutes dissolved in the formulation. It was also observed that the rate of water loss increases with a decrease in tubing ID and a decrease in wall thickness. A relationship that connects the change in protein concentration to a change in tubing weight was also derived from the data and was found to be independent of the protein concentration.

Next, the effect of formulation water activity on the rate of water loss was tested using NaCl and CaCl₂ salt solutions of various weight concentrations, and it was found that high water activity (>0.988) that is typically associated with protein formulations has negligible impact on the rate of water loss. However, for low water activity formulations, the rate of water loss decreases with a decrease in water activity.

The mechanism of water loss was hypothesized to be via diffusion of water through tubing walls in accordance with Fick's 1^{st} law of diffusion. Fick's 1^{st} law was used to calculate the average effective diffusivity of water through tubings containing protein formulations and salt solutions. A relationship between the effective diffusivity and water activity for these solutions was derived. This relationship, along with known formulation properties and tubing parameters, was used to develop a diffusion-based mechanistic model with the goal of capturing experimental water loss data. The applicability of the developed model was tested by studying the experimentally obtained water loss in additional platinum-cured silicone tubings with dimensions not used for deriving the model for several tubing-formulation combinations spanning a wide range of protein concentrations. The experimental water loss was compared with the theoretical upper and lower bounds of water loss derived using the model. It was observed that the experimental water loss for all tubings falls within the bounds calculated using the model, suggesting that the model can successfully explain water loss occurring from formulation-filled platinum-cured silicone tubing for a given formulation and tubing combination.

This work highlights some of the factors that impact the rate of water loss from protein formulation-filled platinum-cured silicone tubing subjected to long holds, and attempts to explain the amount of water lost based on diffusive mass transfer. Note that the environmental factors including temperature and airflow in the tubing surroundings that may impact the rate of water loss require further investigation. Overall, these findings suggest that water loss from silicone tubings and its impact on protein concentration should be taken into consideration during filling holds, small-scale material compatibility studies, and related biopharmaceutical process development and characterization work.

What is claimed is:

1. A method for predicting water loss from a sample including a protein at a time point, comprising:
(a) obtaining a sample including a protein stored in tubing;
(b) using said sample and said tubing to generate a model of water lost from said sample over time in said tubing; and
(c) using said model to predict an amount of water lost from said sample at a time point.

2. The method of claim 1, wherein said model is generated using equations 17, 18 and 20.

3. The method of claim 1, wherein the sample is cell culture fluid (CCF), harvested cell culture fluid (HCCF), any step in the downstream processing of a drug substance (DS), drug substance, or a drug product (DP).

4. The method of claim 1, wherein the tubing internal diameter is known.

5. The method of claim 4, wherein the tubing internal diameter is between about 0.1 mm and about 32 mm, between about 0.2 mm and about 26 mm, between about 0.5 mm and about 16 mm, between about 0.8 mm and about 13 mm, between about 0.8 mm and 10 mm, about 0.1 mm, about 0.2 mm, about 0.3 mm, about 0.5 mm, about 0.6 mm, about 0.8 mm, about 1 mm, about 1.2 mm, about 1.3 mm, about 1.6 mm, about 2.4 mm, about 3.2 mm, about 4.8 mm, about 6.4 mm, about 8 mm, about 9.6 mm, about 12.7 mm, about 15.9 mm, about 19 mm, about 25.4 mm, or about 31.8 mm.

6. The method of claim 1, wherein the tubing thickness is known.

7. The method of claim 6, wherein the tubing thickness is between about 0.1 mm and about 10 mm, between about 0.2 mm and about 8 mm, between about 0.5 mm and about 7 mm, about 0.25 mm, about 0.4 mm, about 0.5 mm, about 0.7 mm, about 0.8 mm, about 1.6 mm, about 1.8 mm, about 2.1 mm, about 2.4 mm, about 3.2 mm, about 4 mm, about 4.8 mm, about 6.4 mm, about 7 mm, about 8 mm, about 9 mm, or about 10 mm.

8. The method of claim 1, wherein the tubing surface area-to-volume ratio is known.

9. The method of claim 8, wherein the tubing surface area-to-volume ratio is between about 0.5 mm⁻¹ and about 5 mm⁻¹, about 0.5 mm⁻¹, about 0.83 mm⁻¹, about 1 mm⁻¹, about 1.25 mm⁻¹, about 1.26 mm⁻¹, about 1.5 mm⁻¹, about 1.54 mm⁻¹, about 1.57 mm⁻¹, about 1.67 mm⁻¹, about 2 mm⁻¹, about 2.11 mm⁻¹, about 2.5 mm⁻¹, about 2.52 mm⁻¹, about 3 mm⁻¹, about 3.1 mm⁻¹, about 3.15 mm⁻¹, about 3.33 mm⁻¹, about 3.5 mm⁻¹, about 4 mm⁻¹, about 4.12 mm⁻¹, about 4.5 mm⁻¹, about 5 mm⁻¹, about 6.25 mm⁻¹, about 6.67 mm⁻¹, about 8.0 mm⁻¹, about 13.33 mm⁻¹, about 20.0 mm⁻¹, or about 40.0 mm⁻¹.

10. The method of claim 1, wherein at least one of the following properties is known: sample volume, concentration of excipients or density.

11. The method of claim 1, wherein the concentration of said protein is known.

12. The method of claim 1, wherein the water activity of said sample is known.

13. The method of claim 12, wherein the water activity is calculated using Equations 12-15.

14. The method of claim 1, further comprising measuring the relative humidity.

15. The method of claim 14, further comprising calculating the average relative humidity using Equation 16.

16. The method of claim 1, wherein the time point is between 10 seconds and 168 hours.

17. The method of claim 1, wherein the tubing vinyl tubing, platinum-cured silicone tubing, peroxide cured silicone tubing, high-density polyethylene (HDPE) tubing, fluoropolymer tubing, or thermoplastic elastomer tubing (TPE).

18. The method of claim 1, further comprising using the calculated water loss at a first time point to iteratively calculate water loss at a later time point at least once.

19. The method of claim 18, wherein said iterative calculation is calculated using Equations 4 and 6.

20. The method of claim 1, wherein a temperature of the tubing is between about 5° C. and about 30° C., between about 15° C. and about 30° C., between about 15° C. and about 25° C., between about 15° C. and about 21° C., between about 16° C. and about 24° C., between about 17° C. and about 23° C., between about 18° C. and about 21° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C.

21. The method of claim 1, wherein the water loss is due to diffusive mass transfer.

22. A method for predicting a change in concentration of a protein at a time point, comprising:
(a) obtaining sample including a protein stored in tubing;
(b) using said sample and said tubing to generate a model of change in concentration of said protein over time in said tubing; and
(c) using said model to predict a change in concentration of said protein at a time point.

23. The method of claim 22, wherein said model is generated using equations 17, 18, 20 and 21.

24. The method of claim 22, wherein the sample is cell culture fluid (CCF), harvested cell culture fluid (HCCF), any step in the downstream processing of a drug substance (DS), drug substance, or a drug product (DP).

25. The method of claim 22, wherein the tubing internal diameter is known.

26. The method of claim 25, wherein the tubing internal diameter is between about 0.1 mm and about 32 mm, between about 0.2 mm and about 26 mm, between about 0.5 mm and about 16 mm, between about 0.8 mm and about 13 mm, about 0.1 mm, about 0.2 mm, about 0.3 mm, about 0.5 mm, about 0.6 mm, about 0.8 mm, about 1 mm, about 1.2 mm, about 1.3 mm, about 1.6 mm, about 2.4 mm, about 3.2 mm, about 4.8 mm, about 6.4 mm, about 8 mm, about 9.6 mm, about 12.7 mm, about 15.9 mm, about 19 mm, about 25.4 mm, or about 31.8 mm.

27. The method of claim 22, wherein the tubing thickness is known.

28. The method of claim 27, wherein the tubing thickness is between about 0.1 mm and about 10 mm, between about 0.2 mm and about 8 mm, between about 0.5 mm and about 7 mm, about 0.25 mm, about 0.4 mm, about 0.5 mm, about 0.7 mm, about 0.8 mm, about 1.6 mm, about 2.1 mm, about 2.4 mm, about 3.2 mm, about 4 mm, about 4.8 mm, about 6.4 mm, about 7 mm, about 8 mm, about 9 mm, or about 10 mm.

29. The method of claim 22, wherein the tubing surface area-to-volume ratio is known.

30. The method of claim 29, wherein the tubing surface area-to-volume ratio is between about 0.5 mm⁻¹ and about 5 mm⁻¹, about 0.5 mm⁻¹, about 0.83 mm⁻¹, about 1 mm⁻¹, about 1.25 mm⁻¹, about 1.26 mm⁻¹, about 1.5 mm⁻¹, about 1.54 mm⁻¹, about 1.57 mm⁻¹, about 1.67 mm⁻¹, about 2 mm⁻¹, about 2.11 mm⁻¹, about 2.5 mm⁻¹, about 2.52 mm⁻¹, about 3 mm⁻¹, about 3.1 mm⁻¹, about 3.15 mm⁻¹, about 3.33 mm⁻¹, about 3.5 mm⁻¹, about 4 mm⁻¹, about 4.12 mm⁻¹, about 4.5 mm⁻¹, about 5 mm⁻¹, about 6.25 mm⁻¹, about 6.67 mm⁻¹, about 8.0 mm⁻¹, about 13.33 mm⁻¹, about 20.0 mm⁻¹, or about 40.0 mm⁻¹.

31. The method of claim 22, wherein at least one of the following properties is known: sample volume, concentration of excipients or density.

32. The method of claim 22, wherein the water activity of said sample is known.

33. The method of claim 32, wherein the water activity is calculated using Equations 12-15.

34. The method of claim 22, further comprising measuring the relative humidity.

35. The method of claim 34, further comprising calculating the average relative humidity using Equation 16.

36. The method of claim 22, wherein the time point is between 10 seconds and 168 hours, between 5 minutes and 168 hours, between 3 hours and 168 hours, between 6 hours and 240 hours, about 10 seconds, about 1 hour, about 6 hours, about 12 hours, about 24 hours, about 36 hours, about 48 hours, about 72 hours, about 96 hours, about 120 hours, about 144 hours, or about 168 hours, or about 240 hours.

37. The method of claim 22, wherein the tubing vinyl tubing, platinum-cured silicone tubing, peroxide cured silicone tubing, high-density polyethylene (HDPE) tubing, fluoropolymer tubing, or thermoplastic elastomer tubing (TPE).

38. The method of claim 22, further comprising using the calculated change in concentration at a first time point to iteratively calculate change in concentration at a later time point at least once.

39. The method of claim 38, wherein said iterative calculation is calculated using Equations 4 and 6.

40. The method of claim 22, wherein a temperature of the tubing is between about 5° C. and about 30° C., between about 15° C. and about 30° C., between about 15° C. and about 25° C., between about 15° C. and about 21° C., between about 16° C. and about 24° C., between about 17° C. and about 23° C., between about 18° C. and about 21° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C.

41. The method of claim 22, wherein the change in concentration is due to water loss from diffusive mass transfer.

42. A method for selecting tubing for a sample including a protein, comprising:
(a) obtaining a sample including a protein stored in at least two tubings;
(b) using said sample and each of said tubings to generate models of water lost from said sample over time in each of said tubings; and
(c) selecting a tubing on the basis of less predicted water loss in step (b).

43. The method of claim 42, wherein said models are generated using equations 17, 18 and 20.

44. The method of claim 42, wherein the sample is cell culture fluid (CCF), harvested cell culture fluid (HCCF), any step in the downstream processing of a drug substance (DS), drug substance, or a drug product (DP).

45. The method of claim 42, wherein the internal diameter of each tubing is known.

46. The method of claim 45, wherein the internal diameter of each tubing is between about 0.1 mm and about 32 mm, between about 0.2 mm and about 26 mm, between about 0.5 mm and about 16 mm, between about 0.8 mm and about 13 mm, about 0.1 mm, about 0.2 mm, about 0.3 mm, about 0.5 mm, about 0.6 mm, about 0.8 mm, about 1 mm, about 1.2 mm, about 1.3 mm, about 1.6 mm, about 2.4 mm, about 3.2 mm, about 4.8 mm, about 6.4 mm, about 8 mm, about 9.6 mm, about 12.7 mm, about 15.9 mm, about 19 mm, about 25.4 mm, or about 31.8 mm.

47. The method of claim 42, wherein the thickness of each tubing is known.

48. The method of claim 47, wherein the thickness of each tubing is between about 0.1 mm and about 10 mm, between about 0.2 mm and about 8 mm, between about 0.5 mm and about 7 mm, about 0.25 mm, about 0.4 mm, about 0.5 mm, about 0.7 mm, about 0.8 mm, about 1.6 mm, about 2.1 mm, about 2.4 mm, about 3.2 mm, about 4 mm, about 4.8 mm, about 6.4 mm, about 7 mm, about 8 mm, about 9 mm, or about 10 mm.

49. The method of claim 42, wherein the surface area-to-volume ratio of each tubing is known.

50. The method of claim 49, wherein the surface area-to-volume ratio of each tubing is between about 0.5 mm⁻¹ and about 5 mm⁻¹, about 0.5 mm⁻¹, about 0.83 mm⁻¹, about 1 mm⁻¹, about 1.25 mm⁻¹, about 1.26 mm⁻¹, about 1.5 mm⁻¹, about 1.54 mm⁻¹, about 1.57 mm⁻¹, about 1.67 mm⁻¹, about 2 mm⁻¹, about 2.11 mm⁻¹, about 2.5 mm⁻¹, about 2.52 mm⁻¹, about 3 mm⁻¹, about 3.1 mm⁻¹, about 3.15 mm⁻¹, about 3.33 mm⁻¹, about 3.5 mm⁻¹, about 4 mm⁻¹, about 4.12 mm⁻¹, about 4.5 mm⁻¹, about 5 mm⁻¹, about 6.25 mm⁻¹, about 6.67 mm⁻¹, about 8.0 mm⁻¹, about 13.33 mm⁻¹, about 20.0 mm⁻¹, or about 40.0 mm⁻¹.

51. The method of claim 42, wherein at least one of the following properties is known:
sample volume, concentration of excipients or density.

52. The method of claim 42, wherein the concentration of said protein is known.

53. The method of claim 42, wherein the water activity of said sample is known.

54. The method of claim 53, wherein the water activity is calculated using Equations 12-15.

55. The method of claim 42, further comprising measuring the relative humidity.

56. The method of claim 55, further comprising calculating the average relative humidity using Equation 16.

57. The method of claim 42, wherein the time point is between 10 seconds and 168 hours, between 5 minutes and 168 hours, between 3 hours and 168 hours, between 6 hours and 240 hours, about 10 seconds, about 1 hour, about 6 hours, about 12 hours, about 24 hours, about 36 hours, about 48 hours, about 72 hours, about 96 hours, about 120 hours, about 144 hours, or about 168 hours, or about 240 hours.

58. The method of claim 42, wherein at least one of the tubings vinyl tubing, platinum-cured silicone tubing, peroxide cured silicone tubing, high-density polyethylene (HDPE) tubing, fluoropolymer or tubing, thermoplastic elastomer tubing (TPE).

59. The method of claim 42, further comprising using the calculated water loss at a first time point to iteratively calculate water loss at a later time point at least once.

60. The method of claim 59, wherein said iterative calculation is calculated using Equations 4 and 6.

61. The method of claim 42, wherein a temperature of each of the tubing is between about 5° C. and about 30° C., between about 15° C. and about 30° C., between about 15° C. and about 25° C., between about 15° C. and about 21° C., between about 16° C. and about 24° C., between about 17° C. and about 23° C., between about 18° C. and about 21° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C.

62. The method of claim 42, wherein the water loss is due to diffusive mass transfer.

63. A method for selecting a range of hold times for a sample in tubing, comprising:
(a) obtaining a sample including a protein stored in tubing;
(b) using said sample and said tubing to generate a model of change in concentration of said protein over time in said tubing; and
(c) selecting a range of hold times on the basis of predicted change in concentration of said protein in step (b).

64. The method of claim 63, wherein a range of hold times is selected to prevent a change in concentration of said protein beyond a determined threshold of percent concentration change.

65. The method of claim 64, wherein said determined threshold of percent concentration change is about 15%, about 10%, about 8%, about 5%, about 2%, or about 1%.

66. The method of claim 65, wherein said determined threshold of percent concentration change is about 10%.

67. The method of claim 63, wherein said model is generated using equations 17, 18, 20 and 21.

68. The method of claim 63, wherein the sample is cell culture fluid (CCF), harvested cell culture fluid (HCCF), any step in the downstream processing of a drug substance (DS), drug substance, or a drug product (DP).

69. The method of claim 63, wherein the tubing internal diameter is known.

70. The method of claim 69, wherein the tubing internal diameter is between about 0.1 mm and about 32 mm, between about 0.2 mm and about 26 mm, between about 0.5 mm and about 16 mm, between about 0.8 mm and about 13 mm, about 0.1 mm, about 0.2 mm, about 0.3 mm, about 0.5 mm, about 0.6 mm, about 0.8 mm, about 1 mm, about 1.2 mm, about 1.3 mm, about 1.6 mm, about 2.4 mm, about 3.2 mm, about 4.8 mm, about 6.4 mm, about 8 mm, about 9.6 mm, about 12.7 mm, about 15.9 mm, about 19 mm, about 25.4 mm, or about 31.8 mm.

71. The method of claim 63, wherein the tubing thickness is known.

72. The method of claim 71, wherein the tubing thickness is between about 0.1 mm and about 10 mm, between about 0.2 mm and about 8 mm, between about 0.5 mm and about 7 mm, about 0.25 mm, about 0.4 mm, about 0.5 mm, about 0.7 mm, about 0.8 mm, about 1.6 mm, about 2.1 mm, about 2.4 mm, about 3.2 mm, about 4 mm, about 4.8 mm, about 6.4 mm, about 7 mm, about 8 mm, about 9 mm, or about 10 mm.

73. The method of claim 63, wherein the tubing surface area-to-volume ratio is known.

74. The method of claim 73, wherein the tubing surface area-to-volume ratio is between about 0.5 mm⁻¹ and about 5 mm⁻¹, about 0.5 mm⁻¹, about 0.83 mm⁻¹, about 1 mm⁻¹, about 1.25 mm⁻¹, about 1.26 mm⁻¹, about 1.5 mm⁻¹, about 1.54 mm⁻¹, about 1.57 mm⁻¹, about 1.67 mm⁻¹, about 2 mm⁻¹, about 2.11 mm⁻¹, about 2.5 mm⁻¹, about 2.52 mm⁻¹, about 3 mm⁻¹, about 3.1 mm⁻¹, about 3.15 mm⁻¹, about 3.33 mm⁻¹, about 3.5 mm⁻¹, about 4 mm⁻¹, about 4.12 mm⁻¹, about 4.5 mm⁻¹, about 5 mm⁻¹, about 6.25 mm⁻¹, about 6.67 mm⁻¹, about 8.0 mm⁻¹, about 13.33 mm⁻¹, about 20.0 mm⁻¹, or about 40.0 mm⁻¹.

75. The method of claim 63, wherein at least one of the following properties is known:
sample volume, concentration of excipients or density.

76. The method of claim 63, wherein the water activity of said sample is known.

77. The method of claim 76, wherein the water activity is calculated using Equations 12-15.

78. The method of claim 63, further comprising measuring the relative humidity.

79. The method of claim 78, further comprising calculating the average relative humidity using Equation 16.

80. The method of claim 63, wherein the time point is between 10 seconds and 168 hours, between 5 minutes and 168 hours, between 3 hours and 168 hours, between 6 hours and 240 hours, about 10 seconds, about 1 hour, about 6 hours, about 12 hours, about 24 hours, about 36 hours, about 48 hours, about 72 hours, about 96 hours, about 120 hours, about 144 hours, or about 168 hours, or about 240 hours.

81. The method of claim 63, wherein the tubing vinyl tubing, platinum-cured silicone tubing, peroxide cured silicone tubing, high-density polyethylene (HDPE) tubing, fluoropolymer tubing, or thermoplastic elastomer tubing (TPE).

82. The method of claim 63, further comprising using the calculated change in concentration at a first time point to iteratively calculate change in concentration at a later time point at least once.

83. The method of claim 82, wherein said iterative calculation is calculated using Equations 4 and 6.

84. The method of claim 63, wherein a temperature of the tubing is between about 5° C. and about 30° C., between about 15° C. and about 30° C., between about 15° C. and about 25° C., between about 15° C. and about 21° C., between about 16° C. and about 24° C., between about 17° C. and about 23° C., between about 18° C. and about 21° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C.

85. The method of claim 63, wherein the change in concentration is due to water loss from diffusive mass transfer.
